(19) United States
(12) Patent Application Publication
(10) Pub. No.: US 2004/0062793 A1
(43) Pub. Date: Apr. 1, 2004

(54) Tissue defect dressings comprising proteinaceous networks

(76) Inventor: Mark Van Dyke, Fair Oaks Ranch, TX (US)

Correspondence Address: PAULA D. MORRIS & ASSOCIATES, P.C., 10260 WESTHEIMER, SUITE 360, HOUSTON, TX, 77042, US

(21) Appl. No.: 10/411,358

(22) Filed: Apr. 10, 2003

Related U.S. Application Data
(60) Provisional application No. 60/393,958, filed on Jul. 5, 2002.

(51) Int. Cl. A61K 38/17; A61L 15/00

(52) U.S. Cl. 424/445; 514/2

(57) ABSTRACT

Proteinaceous tissue defect dressings and methods for their use.

The present application claims the benefit of the filing date of provisional application Ser. No. 60/393,958, filed Jul. 5, 2002. The following are related applications: U.S. patent application Ser. No. 10/119,477, filed Apr. 10, 2002; U.S. patent application Ser. No. 10/127,523, filed Apr. 22, 2002; U.S. patent application Ser. No. 10/133,885, filed Apr. 26, 2002; and, U.S. patent application Ser. No. 10/254,364, filed Sep. 25, 2002.

### FIELD OF THE APPLICATION

The present application relates to tissue defect dressings and to methods for their use. The tissue defect dressings comprise a proteinaceous salve, preferably a proteinaceous film or hydrogel, most preferably a keratinaceous film or hydrogel on a suitable support. Keratinaceous salves are biologically active and encourage correction of tissue defects due to growth factors which stimulate cell growth.

### BACKGROUND OF THE INVENTION

A variety of wound healing preparations are available on the market. Desirable properties for wound healing preparations include the ability to isolate and protect wounds from invasion by infectious agents. A moist dressing is often beneficial. Some of the advantages of a moist wound dressing are: the rehydration of dehydrated tissue; increased angiogenesis, i.e., proliferation of new blood vessels; minimized bacterial growth; physical protection; and the maintenance of the proper pH for stimulating the release of oxygen and for allowing proteolytic enzymes to work more efficiently.

Available wound dressings have certain disadvantages. Powder or granules cannot be applied evenly, do not absorb tissue moisture evenly, and generally are difficult to remove completely from the wound bed. Pastes must be spread onto the tissue. The pressure required to spread the paste can be painful or further traumatize the tissue. In addition, an even application is not always easy to achieve because the product retains its plastic character.

Moist, biocompatible dressings are needed that are easy to apply, easy to maintain in place, easy to remove, and preferably contain factors that actually speed the rate and quality of healing.

### SUMMARY OF THE INVENTION

The present application provides a tissue defect dressing comprising a proteinaceous salve comprising proteinaceous molecules networked by interprotein associations consisting essentially of other than disulfide crosslinks. In a preferred embodiment, the tissue defect dressing comprises a proteinaceous salve comprising keratin molecules comprising interkeratin associations consisting essentially of other than disulfide crosslinks. In a preferred aspect, the proteinaceous salve is selected from the group consisting of a keratinaceous hydrogel and a keratinaceous film.

Where the proteinaceous salve is a hydrogel, the hydrogel comprises water retained in a network comprising keratins comprising interkeratin associations consisting essentially of other than disulfide crosslinks. The interkeratin associations are selected from the group consisting of entanglements, electrostatic bonds, covalent bonds consisting essentially of other than disulfide bonds, and combinations thereof. In a preferred embodiment, the hydrogel comprises water retained in a network consisting essentially of water soluble keratins comprising covalent bonds consisting essentially of other than disulfide bonds, said covalent bonds consisting essentially of interprotein crosslinks comprising first covalent bonds between first functional groups on a plurality of molecules of a crosslinking agent and first reactive pendant groups on a plurality of first water soluble keratins and second covalent bonds between second functional groups on a plurality of molecules of said crosslinking agent and second reactive pendant groups on a plurality of second water soluble keratins.

Where the proteinaceous salve is a film, the film comprises water-soluble proteins consisting essentially of interprotein crosslinks comprising first covalent bonds between first functional groups on a plurality of molecules of a heterogeneous crosslinking agent and first reactive pendant groups on a plurality of first water soluble proteins and second covalent bonds between second functional groups on a plurality of molecules of said heterogeneous crosslinking agent and second reactive pendant groups on a plurality of second water soluble proteins.

The first and second functional groups preferably are selected from the group consisting of alkoxide groups, allyl groups, vinyl groups, hydroxyl groups, amine groups, aldehyde groups, isocyanate groups, ester groups, and anhydride groups. Preferably, the reactive pendant groups are selected from the group consisting of hydroxyl groups, thiol groups, reactive amine groups, and epoxides. Most preferably, at least one of a moiety selected from the group consisting of said first functional groups, said first reactive pendant groups, said second functional groups, and said second reactive pendant groups is an epoxide.

Whether the proteinaceous salve is a hydrogel or a film, the proteinaceous molecules preferably are selected from the group consisting of keratin molecules, collagen molecules, elastin molecules, and combinations thereof. Most preferably, the proteinaceous molecules comprise keratin molecules. In a preferred embodiment, the keratins are derived from human hair. Keratinaceous tissue defect dressings inherently comprise and deliver bioactive components to the tissue defect. The tissue defect dressing also may comprise one or more added bioactive component(s).

The tissue defect dressing preferably further comprises a support adapted to support the proteinaceous salve. The support preferably comprises a sheet of permeable material having an inner surface abutting said proteinaceous salve and an outer surface abutting a barrier material.

In one aspect, the cohesive tissue defect dressing comprises a proteinaceous salve comprising a network comprising water soluble keratins comprising interkeratin crosslinks having the following structure:

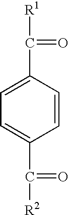

wherein R¹ and R² independently are amino acid residues of separate water soluble proteins, said amino acid residues being selected from the group consisting of cysteine, arginine, serine, lysine, asparagine, glutamine, tyrosine, tryptophan, and histidine.

In another aspect, the cohesive tissue defect dressing comprises a proteinaceous salve comprising a network comprising water soluble keratins comprising interprotein crosslinks having the following general structure:

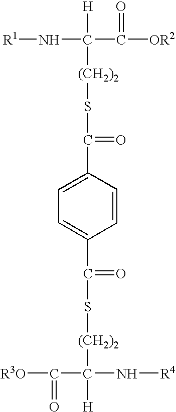

wherein R¹ and R² independently are amino acid residues of separate water soluble proteins, said amino acid residues being selected from the group consisting of cysteine, arginine, serine, lysine, asparagine, glutamine, tyrosine, tryptophan, and histidine.

In yet another aspect, the cohesive tissue defect dressing comprises a proteinaceous salve comprising a network comprising water soluble keratins comprising interkeratin crosslinks having the following general structure:

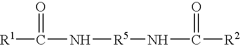

wherein

R¹ and R² independently are amino acid residues of separate water soluble keratins, said amino acid residues being selected from the group consisting of glutamic acid and aspartic acid; and,

R⁵ is selected from the group consisting of alkoxy groups, alkylene groups, and alkenyl groups having from about 1 to about 50 carbon atoms, alone, or in combination with cyclic alkyl groups or aromatic groups.

In yet another aspect, the cohesive tissue defect dressing comprises a proteinaceous salve comprising a network comprising water soluble keratins comprising interkeratin crosslinks having the following general structure:

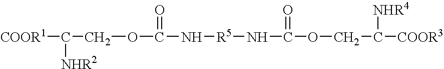

wherein R¹ and R² are the remainder of a first water soluble keratin; and,

R³ and R⁴ are the remainder of a second water soluble keratin.

In another aspect, the cohesive tissue defect dressing comprises a proteinaceous salve comprising a network comprising water soluble keratins comprising interkeratin crosslinks having the following general structure:

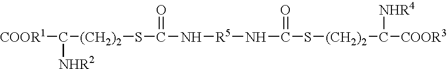

wherein R¹ and R² are the remainder of a first water soluble keratin; and,

R³ and R⁴ are the remainder of a second water soluble keratin.

In another aspect, the cohesive tissue defect dressing comprises a proteinaceous salve comprising a network comprising water soluble keratins comprising interkeratin crosslinks having the following general structure:

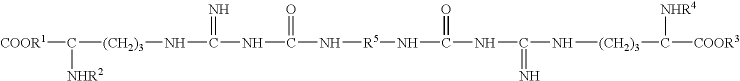

wherein R¹ and R² are the remainder of a first water soluble keratin; and,

R³ and R⁴ are the remainder of a second water soluble keratin.

In another aspect, the cohesive tissue defect dressing comprises a proteinaceous salve comprising a network comprising water soluble keratins comprising interkeratin crosslinks having the following general structure:

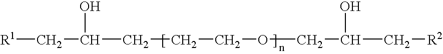

wherein

n is from about 1 to about 50; and,

R¹ and R² independently are amino acid residues of separate water soluble keratins, the residues being selected from the group consisting of cysteine, arginine, serine, lysine, asparagine, glutamine, tyrosine, tryptophan, and histidine.

In another aspect, the cohesive tissue defect dressing comprises a proteinaceous salve comprising a network comprising water soluble keratins comprising interkeratin crosslinks having the following general structure:

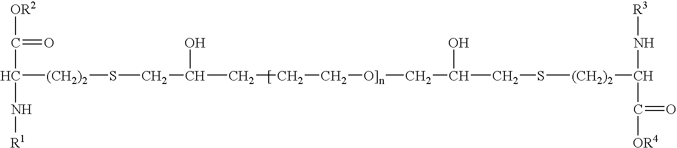

wherein

n is from about 1 to about 50;

R¹ and R² are the remainder of a first water soluble keratin; and,

R³ and R⁴ are the remainder of a second water soluble keratin.

In another aspect, the cohesive tissue defect dressing comprises a proteinaceous salve comprising a network comprising water soluble keratins comprising interkeratin crosslinks having the following general structure:

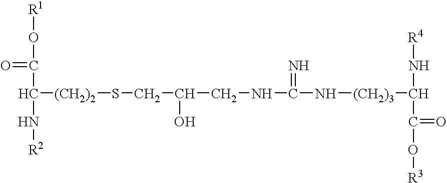

wherein

R¹ and R² are the remainder of a first water soluble keratin; and

R³ and R⁴ are the remainder of a second water soluble keratin.

In another aspect, the cohesive tissue defect dressing comprises a proteinaceous salve comprising a keratinaceous network comprising the following crosslinks:

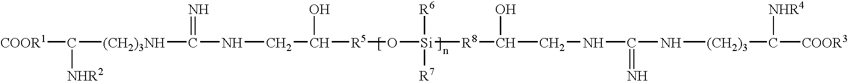

wherein

n is from about 1 to about 50;

R¹ and R²are a remainder of a first keratin molecule;

R³ and R⁴ is a remainder of a second keratin molecule; and,

R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

Preferably,

at least one of R⁶ and R⁷ is an alkyl group, most preferably a methyl group; and,

R⁵ and R⁸ comprise n-propoxypropyl groups.

In another aspect, the cohesive tissue defect dressing comprises a proteinaceous salve comprising a keratinaceous network comprising the following crosslinks:

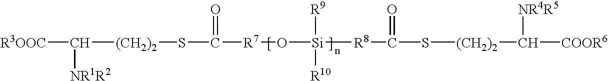

wherein

n is from about 1 to about 50;

R¹, R², and R³ are a remainder of a first protein molecule;

R⁴, R⁵, and R⁶ are a remainder of a second protein molecule; and,

R⁷, R⁸, R⁹ and R¹⁰ are selected from the group consisting of cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

Preferably,

at least one of R⁹ and R¹⁰ is a methyl group; and,

R⁷ and R⁸ preferably are selected from the group consisting of alkyl groups having from about 1 to about 6 carbon atoms and dimethylsiloxy groups.

In another aspect, the cohesive tissue defect dressing comprises a proteinaceous salve comprising a keratinaceous heterogeneous crosslinked network comprising the following crosslinks:

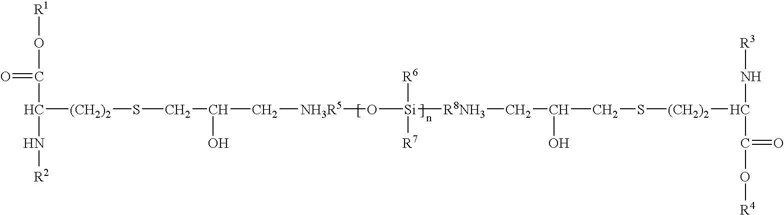

wherein

n is from about 1 to about 50;

R¹ and R² are a remainder of a first keratin molecule;

R³ and R⁴ is a remainder of a second keratin molecule; and,

R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

Preferably,

at least one of R⁶ and R⁷ is an alkyl group, most preferably a methyl group; and

R⁵ and R⁸ comprise n-propoxypropyl groups.

In another aspect, the cohesive tissue defect dressing comprises a proteinaceous salve comprising a keratinaceous heterogeneous crosslinked network comprising the following crosslinks:

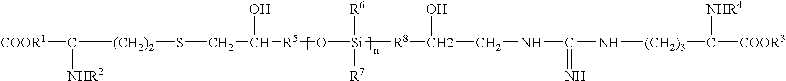

wherein

n is from about 1 to about 50;

R¹ and R² are a remainder of a first keratin molecule;

R³ and R⁴ are a remainder of a second keratin molecule; and,

R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

Preferably,

at least one of R⁶ and R⁷ is an alkyl group, most preferably a methyl group; and,

R⁵ and R⁸ comprise n-propoxypropyl groups.

In yet another aspect, the cohesive tissue defect dressing comprises a proteinaceous salve comprising a keratinaceous heterogeneous crosslinked network comprising the following crosslinks:

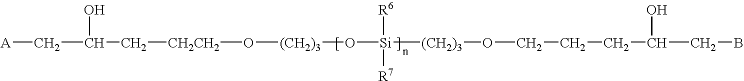

wherein n is from about 5 to about 50; and

A and B are the remainder of first and second keratin molecules.

In another aspect, the cohesive tissue defect dressing comprising a proteinaceous salve comprising a keratinaceous heterogeneous crosslinked network comprising the following crosslinks:

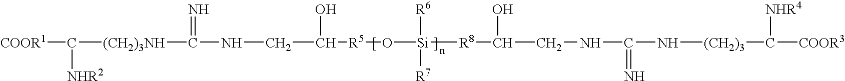

wherein

n is from about 1 to about 50;

R¹ and R² are a remainder of a first keratin molecule;

R³ and R⁴ is a remainder of a second keratin molecule; and,

R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

Preferably,

at least one of R⁶ and R⁷ is an alkyl group, most preferably a methyl group.

In another aspect, the cohesive tissue defect dressing comprises a proteinaceous salve comprising a keratinaceous heterogeneous crosslinked network comprising the following crosslinks:

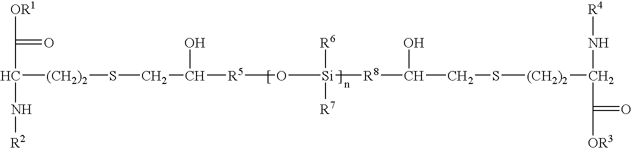

wherein

n is from about 1 to about 50;

R¹ and R² are a remainder of a first keratin molecule;

R³ and R⁴ is a remainder of a second keratin molecule; and,

R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

Preferably,

at least one of R⁶ and R⁷ is an alkyl group, most preferably a methyl group.

In another aspect, the cohesive tissue defect dressing comprises a proteinaceous salve comprising a network comprising proteinaceous molecules comprising interprotein crosslinks consisting essentially of first covalent bonds between first reactive functionalities on a plurality of molecules of a crosslinking agent comprising silicone and first reactive pendant groups on a plurality of first protein molecules and second covalent bonds between second reactive functionalities on a plurality of molecules of said crosslinking agent and second reactive pendant groups on a plurality of second protein molecules. Preferred reactive moieties are selected from the group consisting of epoxy groups and vinyl groups, and preferably comprise the same reactive moiety. In this embodiment, the crosslinking agent preferably has the following general structure:

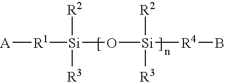

wherein

n is from about 1 to about 50; and,

A and B are the remainder of first and second protein molecules;

at least two of R¹, R², R³, and R⁴ comprise at least one reactive functionality comprising at least one reactive moiety selected from the group consisting of a reactive unsaturated carbon-carbon bond, a reactive oxygen containing group, a reactive nitrogen containing group, and a reactive sulfur-containing group.

More preferably, R¹, R², R³, and R⁴ are selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

Even more preferably,

R¹ and R⁴ independently are selected from the group consisting of hydrogen, linear, branched or cyclic alkyl groups having from about 1 to about 6 carbon atoms, alkenyl groups having from about 2 to about 6 carbon atoms, hydrido groups, alkoxy groups comprising one or more alkyl groups having a total of from about 1 to about 6 carbon atoms, hydroxy groups, alkylamine groups, alkylmercapto groups, acrylate groups, methacrylate groups, halo groups, acetoxy groups, and epoxy groups; and,

R² and R³ independently are selected from the group consisting of hydrogen, cycloalkyl groups, vinyl groups, hydrido groups, trifluoroalkyl groups, phenyl groups, alkyl groups, alkoxy groups, alkylmercapto groups, and alkylamine groups; provided that, when one of R² or R³ is a vinyl group, the other of R² or R³ is a group other than a hydrido group; and, when one of R² or R³ is a hydrido group, the other of R² or R³ is a group other than a vinyl group.

Most preferably,

at least one of R² and R³ is an alkyl group, most preferably a methyl group.

### BRIEF DESCRIPTION OF THE FIGURES

FIG. 1 is an amino acid analysis of reductively extracted low molecular weight keratins, specifically sample 4-AKR-112-2A from Example 3.

FIG. 2 is a cone and plate viscosity analysis of keratin hydrogel made with 10 weight percent of sample no. 4-AKR-112-2B from Example 5.

FIG. 3 is a graph of the storage (G′) and loss (G″) dynamic shear moduli of a keratin hydrogel made with 10 weight percent of sample no. 4-AKR-112-2B from Example 5.

FIG. 4 is a graph of the dynamic shear viscosity of non-crosslinked keratin hydrogels from Example 5.

FIG. 5 is a graph of the dynamic shear viscosity of DER 332 resin crosslinked keratin hydrogels of Example 5.

FIG. 6 is a graph of the dynamic shear viscosity of three crosslinked keratin hydrogels of Example 5.

FIG. 7 is a graph of the tan d measurement of non-crosslinked and DER 332 resin crosslinked keratin hydrogels of Example 5.

FIG. 8 is a photograph from the in vitro scratch assay used in wound studies.

FIG. 9 is an illustration of the wound placement pattern in the porcine model.

FIG. 10 is a chart of the percentage not healed vs. dilutions per milligram from the fibroblast cultures of Example 14.

FIG. 11 is a chart of the wound area after 24 hours (mm²) vs. dilutions per microgram for the A-431 cell cultures of Example 14.

FIGS. 12-16 are histological impressions of demonstrating the hypertrophic response of the epidermis for hydrogels and films vs. controls.

FIG. 17 is a cross-sectional view of a wound dressing along line A-A in FIG. 17A.

FIG. 18 is a graph illustrating re-epithelialization of keratin treated wounds in a porcine model compared to an occlusive dressing control (Example 16).

FIG. 19 is a graph illustrating neovascularization of keratin treated wounds in a porcine model compared to an occlusive dressing control (Example 16).

### DETAILED DESCRIPTION OF THE INVENTION

The present application provides tissue defect dressings comprising a proteinaceous salve, and provides methods for treating tissue defects using these dressings.

As used herein, the phrase “tissue defect,” includes, but is not necessarily limited to wounds, ulcers, burns, natural defects, such as birth defects, and any other defects of bodily tissue, including skin, bone, cartilage, gastrointestinal organs, and other internal organs. The term “wound” refers to damage to any tissue in a living organism. A wound may be in a soft tissue, such as the spleen, or in a hard tissue, such as bone. A wound may have been caused by any agent, including traumatic injury, disease, infection, surgical intervention, or natural causes.

Preferred tissue defects for treatment with the dressing described herein are skin defects. Examples of treatable skin defects include, but are not necessarily limited to dry skin, eczema, dermatitis, psoriasis, acne, hives, athlete's foot, impetigo, cellulitis, abrasions, incisions, punctures, skin cancers, cysts, rashes, keloid scars, hypertrophic scars, birth defects, and cosmetic defects, such as wrinkles.

Certain proteins, such as keratin, are inherently biocompatible. Elastomeric films and hydrogels comprising keratin networks have been made and have proven useful and effective to treat tissue defects. Foams comprising keratin networks also have been made. The tissue defect dressing of the present application comprises a biocompatible “proteinaceous salve” which is applied directly to the tissue defect. The “proteinaceous salve” comprises a proteinaceous network, preferably a keratinaceous network, which is integral with a suitable carrier. The salve may take a variety of forms, including but not necessarily limited to a film, a hydrogel, a foam, and the like. The proteinaceous network comprises “proteinaceous molecules,” selected from the group consisting of peptides and protein molecules, comprising intermolecular associations consisting essentially of other than disulfide bonds. “Consisting essentially of other than disulfide bonds” is not intended to exclude the presence of disulfide bonds altogether, but to indicate that the type of associations crosslinking the protein/peptide molecules are primarily other than disulfide bonds.

It is possible to apply the proteinaceous salve in bulk form, i.e., by applying or spreading the proteinaceous salve, alone, onto the wound. However, the tissue defect dressings preferably are “cohesive,” defined as comprising a substantially solid but elastic and pliable, self-supporting structure. It is possible to achieve a “cohesive” structure by manipulating the type and quantity of interprotein/interpeptide bonds in the proteinaceous network to increase the strength of the proteinaceous network, itself. In a preferred embodiment, a cohesive tissue defect dressing is achieved by permeating the proteinaceous salve into and/or bonding the proteinaceous salve onto a suitable support.

The support

In a preferred embodiment, the tissue defect dressing comprises a support. The support may be substantially any material adapted to provide structure so that the dressing can be handled easily and which also serves as a semi-permeable barrier to moisture and keeps out pathogens. The precise structure of the support may vary depending upon the type of tissue defect. Many materials are used for this purpose and the choice of support can be made such that an optimal level of moisture retention and protection is afforded. See Acute & Chronic Wounds: Nursing Management, 2^{nd} Edition, Ruth A. Bryant (editor), Mosby Inc., St. Louis (2000), incorporated herein by reference.

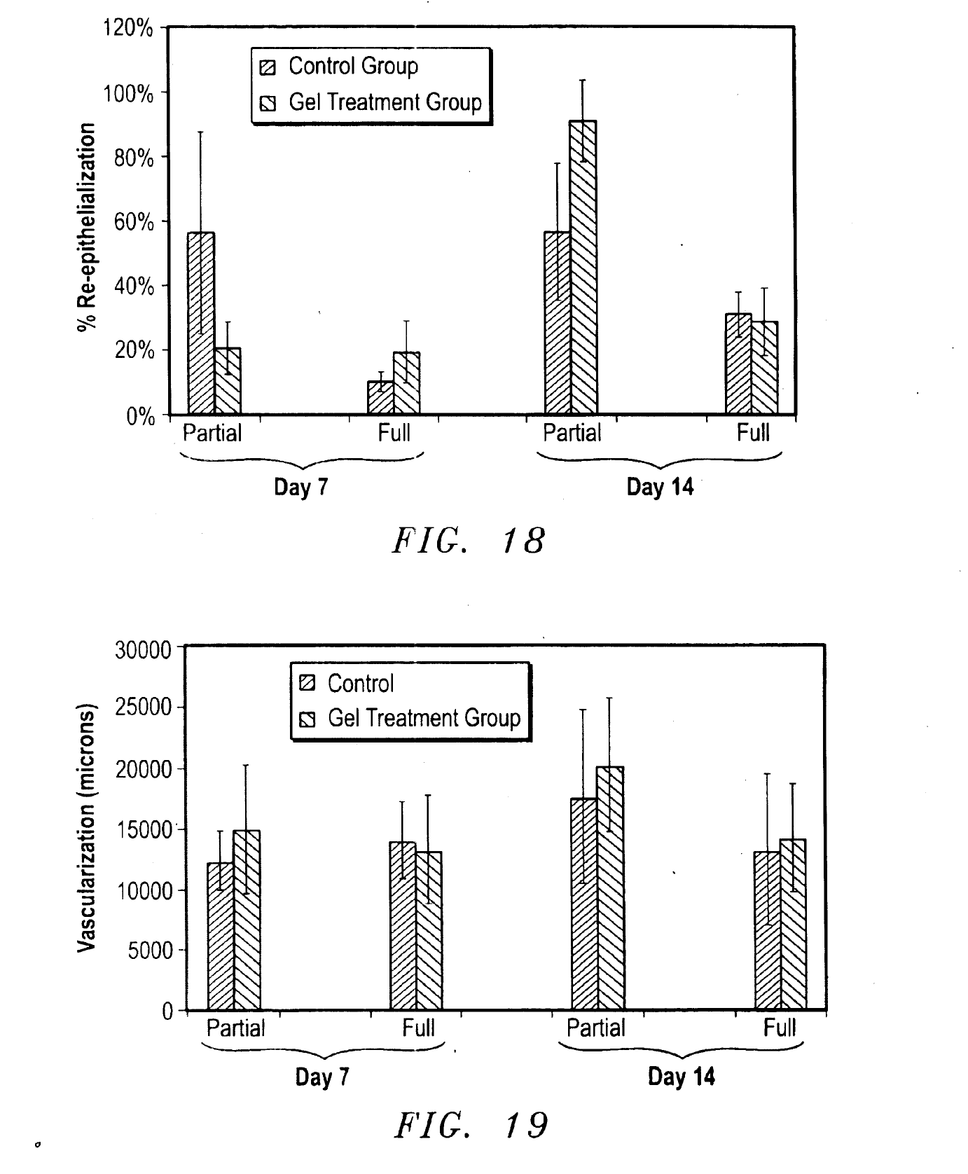

In a preferred embodiment (FIG. 17), the tissue defect dressing 10 comprises a proteinaceous salve 14 and a support 12 comprising a permeable layer 13, a bonding region 16, and a barrier layer 20. In a preferred embodiment, the support 12 is a substantially flat or planar sheet (FIG. 17A).

The permeable layer 13 preferably comprises a relatively porous material adapted to permit only a portion of the abutting proteinaceous salve 14 to permeate the permeable layer 13. Suitable materials for the permeable layer 13 include, but are not necessarily limited to permeable synthetic polymers, permeable natural fibers, and combinations thereof. A most preferred permeable layer 13 comprises cotton. The bonding region 16 may comprise any conventional material for bonding a gel material to a support material. For example, the bonding region 16 may comprise a biocompatible adhesive. In a preferred embodiment, the bonding region 16 comprises at least a portion of the proteinaceous salve 14 permeated into pores in the abutting surface 17 of the permeable layer 13. At an opposed surface 20 of the permeable layer 13 is a barrier material 20 adapted to provide a controlled moist environment, allowing moisture to permeate the tissue defect if it becomes too dry and also allowing moisture to escape from the tissue defect if it becomes too wet. Suitable barrier materials include, but are not necessarily limited to semipermeable adhesive films, perforated films, natural or synthetic cloth, nonwoven films and webs, and extruded films. The support used in the examples is a TELFA® pad (Beiersdorf, Inc., Wilton, Conn.) from which the inner polymer lining is removed to expose the inner cotton core which is bonded to a keratinaceous hydrogel.

The tissue defect dressing 12 preferably is enclosed in a conventional casing and sterilized using conventional means. For example, the tissue defect dressing may be enclosed in a porous paper sheet which permits entry of a sterilizing gas, such as ethylene oxide, but is impervious to pathogenic organisms. Exposure to ethylene oxide for a period of about 360 minutes generally is satisfactory for sterilization. Alternately, the material may sterilized by exposure to ionizing radiation from a gamma radiation source. An exemplary paper sheet is 37.5-pound per ream porous, waterproof paper, e.g. TYVEK™ paper (available from DuPont, Inc. of Wilmington, Del.). The proteinaceous salve 14 preferably is protected by a conventional plastic sheet 22, which may be simply peeled off before application. An example of a suitable the plastic sheet is a 5 mil laminate, e.g. of polyethylene, aluminum foil and MYLAR™ as available from Technipaq Corporation of Chicago, Ill. Where a TELFA® pad is used, the inner polymer lining may be reapplied to the surface of the proteinaceous salve, and the structure encased and sterilized. Various structures may be provided at one end of the pouch or casing to facilitate opening.

In order to treat a tissue defect, the surface of the proteinaceous salve is applied directly to the tissue defect. The tissue defect dressing is strong enough to allow for easy removal and to provide some cushioning for the wound bed, i.e., protecting the wound. The dressing, particularly the hydrogel, contains a large quantity of moisture that will maintain the wound in a moist condition. The dressing also absorbs exudate from the wound as well as evaporates moisture from its top surface. The dressing will remain in place after application but can be easily removed when required.

Delivery of Bioactive Components

The hydrogels and heterogeneous films are shown herein to serve as a delivery vehicle for bioactive components present in the keratin, itself. The hydrogels and films also are useful as a delivery vehicle for other therapeutic and bioactive components, including but not necessarily limited to growth factors, cytokines, chemotactic agents, pharmaceuticals, proteins, cells, and extracellular components. In order to prepare hydrogels or films containing such bioactive components, the bioactive components are simply added to the solution used to form the hydrogel or film.

Keratin

A large variety of proteins and/or peptides may be used to form the networks in the proteinaceous salve. The proteins and/or peptides preferably have a molecular weight of at least about 10 kDa, more preferably at least about 40 kDa, and most preferably about 60 kDa or more. The proteins are biocompatible, with the result that they inherently provide control over biological compatibility. Examples of suitable naturally occurring proteins include, but are not necessarily limited to proteins derived from keratin, collagen, and elastin. The proteins may be derived from natural, synthetic, or recombinant sources. A preferred source of keratin proteins is hair or fur. The hair may be animal, or human. Preferred proteins are relatively high in cysteine content. Most preferred proteins are keratins, preferably water soluble keratins, even more preferably water soluble α-keratins, most preferably water soluble high molecular weight keratins (HMWK's).

Keratins are loosely defined as the hardened and insolubilized proteins found in the epidermal cells of vertebrates. Human hair is composed almost entirely of keratins. Human hair has a cuticle, which is a tough tubular outer layer made up of flattened cells arranged in a scaly, overlapping profile. The inner bulk of the hair is called the cortex and is constructed from elongated cells that are densely packed with fibrous keratins. The fibrous keratins are arranged in bundles referred to as microfibrils and possess an α-helical tertiary structure. The microfibrils are bound together with an amorphous keratin matrix.

The amorphous keratin matrix and the microfibrils vary in function and composition. The matrix is the “glue” that holds the microfibrils together. This matrix “glue” is high in sulfur content, and is comprised of low molecular weight keratins (LMWK) which typically have an average molecular weight of from about 10 to about 15 kDa. The microfibrils are comprised of α-keratins, including high molecular weight keratins (HMWK), having a relatively lower sulfur content, but having a higher average molecular weight of typically from about 50 to about 85 kDa. HMWK's and LMWK's vary in chemical properties, such as reactivity and solubility.

Keratins are afforded their structural integrity, in large part, by the presence of disulfide crosslinks which form a three dimensional network of polyprotein chains. This network structure renders keratins insoluble. Keratins can, however, be made water soluble by destroying this three dimensional structure via disulfide bond scission. Disulfide bond scission can be performed either oxidatively, reductively, or using some combination of both types of bond scission. Oxidative bond scission with hydrogen peroxide, for example, results in the formation of sulfonic acid residues produced from cystine. The material produced using hydrogen peroxide for disulfide bond scission is highly ionic and has excellent water solubility. Reductive bond scission with mercaptoethanol, for example, results in the formation of cysteine residues produced from cystine. The material produced using this reductive technique is highly reactive and will readily re-crosslink.

Hydrogels

In a preferred embodiment, the proteinaceous salve is a hydrogel comprising a proteinaceous network derived from keratin. Hydrogels are networks of hydrophilic macromolecules that swell in water, but do not dissolve. Hydrogels are used in a variety of medical applications. The network of hydrophilic macromolecules can comprise polymer entanglements (virtual crosslinks), pseudo crosslinks, and/or covalent crosslinks. The point at which the molecular weight of a given macromolecule is sufficient for virtual crosslink formation is known as the critical entanglement molecular weight (MW_{c}). At the MW_{c}, a virtual network is formed, the molecular weight of the material is essentially infinite, and the viscosity increases exponentially as the material reaches its gel point. The macromolecules form pseudo crosslinks when a coordinating molecule attaches itself to two adjacent macromolecules, thus forming a “bond” between them. These types of “bonds” typically are electrostatic in nature, the most common being ionic and hydrogen bonds. Covalent crosslinks typically form the most stable networks.

Regardless of whether the network crosslinks are virtual, pseudo, and/or covalent, the hydrogel resists dissolution because of the nominally infinite molecular weight resulting from the crosslinks. The type of crosslinks found in the network controls the mechanical characteristics of the resulting hydrogel, as well as the in vivo degradation rate as the network is acted upon at the cellular and molecular level.

Hydrogels have the advantage that they can have a relatively high solids content while retaining a high level of biocompatibility. The proteinaceous networks preferably comprise water soluble proteins, preferably water soluble keratins, crosslinked in a manner that affords broad control over mechanical, chemical, and biological properties. The crosslinking preferably consists essentially of hydrolyzable and non-hydrolyzable crosslinks to provide control over biodegradability.

In order to make a hydrogel using human hair, sufficiently high molecular weight α-keratins from within the microfibrils are selectively extracted and isolated, preferably as described above. Although the exact MW_{c} for HMWK's is not known, the molecular weight of HMWK's is sufficiently high for virtual crosslink formation and creation of a three-dimensional keratin network. The keratins in the network are hydrophilic in nature. As a result, the three-dimensional keratin network may be swelled with water to produce a hydrogel. Gelation and swelling can be enhanced by the presence of ions. The keratin network is able to sequester the ions, which form pseudo crosslinks and make the network even more hydrophilic. These hydrogels are preferred for some applications.

Unfortunately, hydrogel networks formed solely of critical entanglements and/or pseudo crosslinks may not be sufficiently stable for many uses since the proteins in such hydrogel networks are not covalently linked. In order to produce truly stable hydrogels that exhibit little to no “creep,” covalent crosslinks must be introduced into the hydrogel.

The present application describes suitable covalent crosslinks for forming more stable proteinaceous, preferably keratinaceous hydrogels, and provides methods for forming proteinaceous networks comprising such covalent bonding. The hydrogels comprising covalent crosslinks may be engineered to produce a predetermined rate of biodegradation. The rate of biodegradation is controlled by (a) providing hydrolyzable and/or non-hydrolyzable covalent bonding between the proteins in the hydrogel; and (b) manipulating the accessibility of the covalent crosslinks to water molecules.

Covalent bonds that are susceptible to hydrolytic cleavage, such as ester or ether bonds, can be used to impart a relatively rapid biodegradation rate. Conversely, hydrolytically stable bonds such as carbon-carbon, carbon-nitrogen, and carbon-sulfur bonds can be used to extend in vivo biostability. The accessibility of the crosslink to water molecules is manipulated by the introduction of steric hindrance, preferably by flanking susceptible bonds with hydrophobic groups to impart hydrolytic stability.

Disulfide bond scission and keratin extraction

The following methods are preferred for processing keratins to produce hydrogels. Although the hydrogels may be made using other proteins, the following description refers to keratins for the sake of simplicity.

The keratins may be processed and/or isolated in a number of ways. Preferably, the processing is sufficient to render the resulting proteins water soluble. Suitable processing techniques include known oxidation techniques, reductive techniques, and/or combinations thereof, as long as the processing renders the proteins water soluble without significant hydrolysis of peptide bonds. At least in the case of keratins, preferred proteins are HMWK's which are processed and isolated by a two step reduction process.

A number of reductive chemistries are known for disulfide bond scission in keratins: See Wardell, J. L., “Preparation of Thiols” in The Chemistry of the Thiol Group, Patai, S. (Editor), pp. 163-353, John Wiley & Sons, New York, N.Y. (1974), incorporated herein by reference. HMWK's may be extracted from hair using at least two reductive extractions, as described in Crewther, W. G., Fraser, R. D. B., Lennox, F. G., and Lindley, H., “The Chemistry of Keratins” in Advances in Protein Chemistry, Anfinsen, C. B., Jr., Anson, M. L., Edsall, J. T., and Richards, F. M. (Editors), Academic Press, New York, pp. 191-346 (1965), incorporated herein by reference.

In a preferred embodiment, a first reductive extraction is performed by treating the hair with a first reducing agent under first conditions effective to selectively extract matrix keratins, producing a first solution comprising soluble reduced matrix keratins (LMWK's) and remaining hair solids (HMWK's). Although it may be possible to subject the LMWK's to the techniques described herein to produce hydrogels, preferred proteins for use in the techniques herein are HMWK's, which preferably are isolated during a second extraction. The remaining hair solids and the first solution are separated, and the remaining hair solids are exposed to a second extraction solution under second conditions effective to solubilize α-keratins, producing a second solution comprising soluble reduced α-keratins (HMWK's) and solid cuticle.

Suitable reducing agents include, but are not necessarily limited to thioglycolic acid and salts thereof, mercaptoethanol, dithiothreitol, thioglycerol, thiolactic acid, glutathione, cysteine, sodium sulfide, and sodium hydrosulfide. Preferred reducing agents are thioglycolic acid and mercaptoethanol, most preferably thioglycolic acid.

In order to selectively reduce and extract the desired proteins, the hair (or other protein source) is suspended in a reducing agent at a concentration of from about 0.1M to about 10M, preferably about 1.0M. Gentle swelling of hair fibers is achieved at a pH of about 9 or more, preferably at a pH of from about 9 to about 10.5. Hence, the initial reduction takes place at a temperature of from about 20 to about 100° C., preferably at about 25° C. The time period required to accomplish the first reduction is from about 4 to about 24 hours, most preferably about 12 hours. The reaction occurs under an inert atmosphere, preferably nitrogen. The liquid fraction is separated from remaining solids using known means, including but not necessarily limited to filtration, cannulation, and/or centrifugation, preferably under inert atmosphere. A preferred method of separation is filtration.

A second extraction is performed using a suitable swelling agent, preferably urea, bases such as ammonium hydroxide, sodium hydroxide, or potassium hydroxide. A most preferred swelling agent for this second extraction is concentrated urea. The second extraction effectively removes the fibrous α-keratins from inside the cuticle. The second extraction occurs at from about 1M to about 10M urea, preferably about 7M urea, for a period of at least about 1 hour, preferably from about 1 to about 72 hours, most preferably about 24 hours. The second extraction occurs at room temperature, but may take place at temperatures of from about 20° C. to about 100° C., preferably about 25° C. The liquid fraction is separated from the empty, intact cuticle, using known means. Suitable means include but are not necessarily limited to filtration, cannulation and/or centrifugation, preferably under inert atmosphere. A preferred method of separation is filtration.

Once the cuticle is removed, the water soluble keratin proteins may be retained in solution for further use, or they may be isolated from the solution by addition to a water-miscible non-solvent, or by spray drying. Water-miscible non-solvents include, but are not necessarily limited to ethanol, methanol, isopropyl alcohol, tetrahydrofuran, acetone, dioxane, and the like, again under inert atmosphere. A preferred non-solvent is ethanol. The precipitate is separated from the non-solvent using known means, preferably by filtration and rinsing using additional aliquots of the non-solvent. The precipitated proteins are dried using known techniques, preferably overnight under vacuum at room temperature. The extracted water soluble keratin proteins (herein sometimes collectively referred to as “water soluble proteins”) comprise thiols or thiol groups.

Thiols possess reactivities similar to alcohols, and can be used to perform a multitude of known organic chemical reactions, such as those described in McMurry, J., Organic Chemistry, Brooks/Cole Publishing Co., Monterey, Calif. (1984); Scudder, P. H., Electron Flow in Organic Chemistry, John Wiley & Sons, New York, N.Y. (1992); Stowell, J. C., Intermediate Organic Chemistry, John Wiley & Sons, New York, N.Y. (1994), incorporated herein by reference. The rate of reduction is affected by reagent concentration(s), reaction temperature(s), and exposure time(s).

Formation of Covalent Crosslinks

Thiols and other chemical moieties contained in amino acid residues have utility as labile sites for covalently crosslinking the water soluble proteins. Preferred reactions are reactions that form other than disulfide bonds.

In a preferred embodiment, crosslinking agents are used to produce covalent bonding. Preferred crosslinking agents produce biocompatible byproducts, preferably hydrogen, water, carbon dioxide, and/or any other biocompatible byproduct that is readily metabolized or excreted, removed from the network, or at least is not toxic to the human body.

In order to prepare the networks, the desired crosslinking agent(s) are determined. Crosslinking agents other than glutaraldehyde having two or more of the same functional groups, or two or more different functional groups are suitable. Preferable crosslinking agents have two or more of the same functional group, as described below.

In a preferred embodiment, the HMWKs are provided as a dry powdered precursor. If a crosslinking agent is used, the crosslinking agent is provided in an aqueous solution which is added to the powdered HMWKs. Upon mixing, a viscous hydrogel results. The aqueous solution of crosslinker is prepared such that at least about 5 wt. %, preferably about 10 wt. %, relative to the keratin, of the multifunctional crosslinking agent is added, forming a hydrogel. Depending upon the crosslinking agent, a catalyst or promoter may be added. The hydrogel may also be formed first by adding the water to the HMWK powder, followed by adding the crosslinking agent and a catalyst.

Crosslinking reactions

Crosslinking of the water soluble proteins and network formation occurs, generally, when a non-reactant which is at least difunctional, or has at least two reactive groups, is used to crosslink between reactive pendant groups on two different water soluble keratin proteins. The non-protein reactant creates a bridge between the water soluble proteins, thereby producing a three-dimensional network.

Proteins comprise amino acids, which generally have the formula:

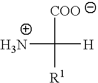

Table 1 summarizes the amino acid residues found in human hair, for example, and shows the “R¹” groups associated with each residue.

TABLE 1
Ranked average amounts of amino acids in human hair

| | | | | Isoelectric | Percent |
|---|---|---|---|---|---|
| | | | | Point | Composition |
| Amino Acid | R¹ Group | Nature | pKa | (pH) | in Hair |
| Cysteine | 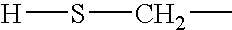 | Nonpolar | 8.4 | 5.02 | 17.3 |
| Glutamic Acid | 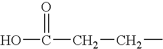 | Polar | 4.5 | 3.22 | 13.9 |
| Arginine | 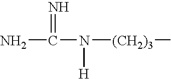 | Polar | 12.5 | 11.15 | 9.85 |
| Serine | 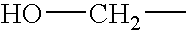 | Polar | None | 5.68 | 9 |
| Threonine | 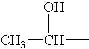 | Polar | None | 5.64 | 7.75 |
| Leucine | 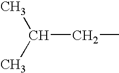 | Hydro- phobic | None | 5.98 | 7.35 |
| Proline | 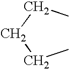 | Hydro- phobic | None | 6.3 | 6.95 |
| Aspartic Acid | 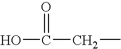 | Polar | 4.5 | 2.77 | 5.8 |
| Valine | 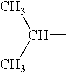 | Hydro- phobic | None | 5.96 | 5.7 |
| Isoleucine | 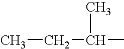 | None | 5.94 | 4.75 | |
| Glycine | 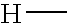 | Nonpolar | None | 5.65 | 4.15 |
| Phenylalanine | 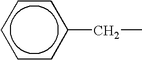 | Hydro- phobic | None | 5.48 | 3 |
| Alanine | 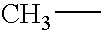 | Hydro- phobic | None | 6 | 2.8 |
| Tyrosine | 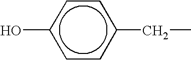 | Hydro- phobic | None | 5.66 | 2.6 |
| Lysine | NH₂—(CH₂)₄— | Polar | 10.4 | 9.59 | 2.5 |
| Histidine | 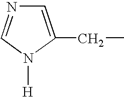 | Aromatic | 6.2 | 7.47 | 0.9 |
| Methionine | 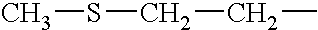 | Hydro- phobic | None | 5.74 | 0.85 |
| Tryptophan | 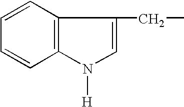 | Hydro- phobic | None | 5.89 | 0.85 |

The most abundant amino acid in human hair is cysteine, which is found in the form of disulfide-bridged cystine groups. As discussed above, this group can be converted to other sulfur containing moieties, most notably thiol. Thiols theoretically can be reacted with reactive ends of a crosslinking agent using a number of chemical techniques, such as those described in S. Patai (Ed.), the Chemistry of the Thiol Group, Parts 1 and 2, John Wiley & Sons, New York, N.Y. (1974), incorporated herein by reference. Other reaction scenarios, such as those directed toward polymer synthesis, also are useful to utilize thiols to form an assortment of desirable crosslinks, including those described in Rempp, P. and Merrill, E. W., Polymer Synthesis, Huethig & Wepf Verlag Basel, Heidelberg, Germany (1986); Young, R. J. and Lovell, P. A., Introduction to Polymers, Chapman & Hall, London (1991); Odian, G., Principles of Polymerization, John Wiley & Sons, New York, N.Y. (1991), incorporated herein by reference.

In addition to cysteine, the following amino acids have pendant groups comprising nitrogen or oxygen which may be useful as reactive pendant groups; arginine, serine, glutamic acid, threonine, aspartic acid, lysine, asparagine, glutamine, tyrosine, tryptophan, and histidine. Where the protein is α-keratin, preferred amino acid residues comprising reactive pendant groups for crosslinking are cysteine, arginine, serine, and glutamic acid, most preferably cysteine and arginine.

Crosslinking agents comprise at least two reactive groups. Preferred reactive groups are selected from the group consisting of epoxide groups, isocyanate groups, and carboxyl groups. Most preferred crosslinking agents are diepoxides, diisocyanates, and dicarboxylates, including anhydrides and hydrolyzed diacids thereof.

Other suitable crosslinking agents include, but are not necessarily limited to polyethylene glycols (PEGs), multi-arm PEGs, and polyethylene glycol derivatives. Suitable polyethylene glycol derivatives include, but are not necessarily limited to nucleophilic PEGs; electrophilically active PEGs, heterofunctional PEGs, PEG-biotins, vinyl derivatives, and PEG phospholipids. Exemplary crosslinking agents include, but are not necessarily limited to: mPEG-acetaldehyde diethyl acetal; mPEG-acrylate; mPEG2-aldehyde; mPEG-amine; ω-amino-α-carboxyl PEG; mPEG-benzotriazole carbonate, PEG-biotin, t-Boc-protected amine; mPEG-double esters; fluorescein-PEG-NHS; FMOC protected amine; mPEG forked maleimide; mPEG maleimide; multi-arm PEG; NHS-PEG- maleimide; NHS-PEG-vinlsulfone; mPEG2-N-hydroxysuccinimide; PEG-phospholipid; mPEG propionaldehyde; mPEG succinimidyl butanoate, and mPEG succinimidyl proprionate, available from Shearwater Corporation, Huntsville, Ala.

For convenience, the crosslinking agents described herein sometimes are referred to as “di-”functional. However, unless a crosslinking agent is expressly claimed or expressly stated to be di-functional only, it is to be understood that the crosslinking agents described herein may also be multi-functional, e.g., di-, tri, tetra-, etc. The non-functional portion of the molecule (R⁵, below) generally forms the remainder of the “bridge” crosslinking the proteins. R⁵ is biocompatible and typically is an organic moiety. Suitable organic moieties include, but are not necessarily limited to alkoxy groups, alkylene groups, and alkenyl groups having from about 1 to about 50 carbon atoms. The alkoxy groups, alkylene groups, or alkenyl groups may be present alone, or in combination with cyclic alkyl groups or aromatic groups.

Without limiting the claims to a particular theory or mechanism of action, unless expressly claimed, the following are crosslinking chemistries involved in producing the crosslinked water soluble protein networks:

Addition to Amine Groups

A preferred, covalently crosslinked hydrogel is made by addition reactions between reactive amine groups and oxirane compounds. Such addition reactions occur readily without the aid of a catalyst. The crosslinking reaction with arginine is as follows:

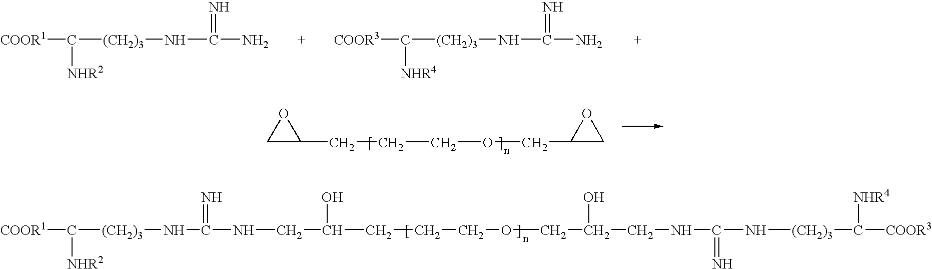

wherein R¹ and R² represent the remainder of one water soluble protein molecule and R³ and R⁴ represent the remainder of a separate water soluble protein molecule, preferably different water soluble α-keratin molecules.

In order to perform this reaction, the water soluble keratins are exposed to a solution containing an oxirane-containing aliphatic or aromatic compound, typically at a concentration of up to about 20 weight percent relative to keratin, preferably between 5 and 10 weight percent relative to keratin; at a pH between about 4 and 9, preferably about 7; at a temperature of from about 0 to about 100° C., preferably about 37° C., preferably for a time period of from about 0 to about 72 hours, most preferably about 24 hours. This process results in the formation of a hydrogel within 1 to 2 minutes and the crosslinking reaction occurs while the keratins are in the gelled state.

A preferred oxirane compound is a diepoxide having the following general structure:

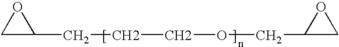

wherein n is from about 1 to about 50. Preferred epoxides include DER™332 and DER™736, available from the Dow Chemical Company.

A similar reaction occurs when a diepoxide reacts with cysteine residues:

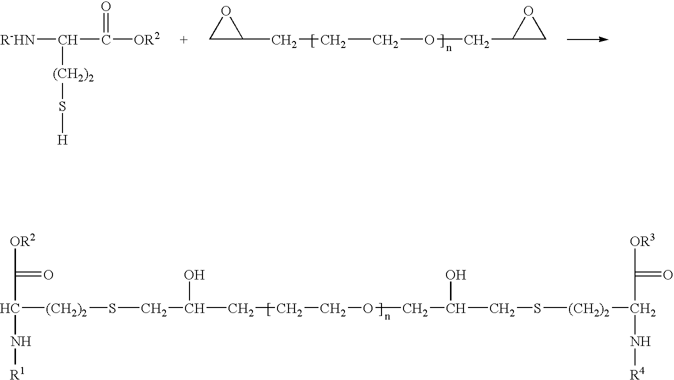

wherein n is from about 1 to about 50, R¹ and R² are the remainder of a first water soluble protein molecule and R³ and R⁴ are the remainder of a second water soluble protein molecule.

Conversion of thiol by condensation

Condensation reactions such as transesterification, for example, can be used to generate thioesters. An example of a transesterification reaction is shown below:

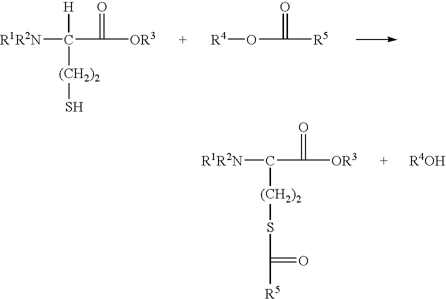

wherein R¹ and R² comprise entities selected from the group consisting of hydrogen and the remainder of the N-terminal portion of the protein molecule; R³ comprises the remainder of the carboxy-terminal portion of the protein molecule; R⁴ is an appropriate leaving group; and, R⁵ is a functional hydrocarbon. Suitable R⁴ groups include, but are not necessarily limited to hydrogen, alkyl groups having from about 1 to 6 carbon atoms, and aryl groups, including benzyl groups. Suitable R⁵ groups include, but are not necessarily limited to aryl groups, including benzyl groups, and alkyl and allyl groups having from about 1 to about 20 carbon atoms in combination with any number of heteroatoms, such as oxygen and nitrogen, and polyalkylethers comprising from about 1 to 50 repeat groups.

In order to use this reaction to crosslink water soluble keratins, a hydrogel comprising from about 1 wt. % to about 20 wt. % water soluble keratins is exposed to a multi-ester or an anhydride of a multi-ester. Suitable multi-esters include, but are not necessarily limited to diesters having from about 1 to 3 carbon atoms (methyl, ethyl, and propyl diesters, respectively) of desired alkyl and aryl carboxylic acids. A preferred embodiment uses phthalic anhydride, which is hydrolyzed to phthalic acid. The exemplary reaction with terephthalic acid is believed to proceed as follows:

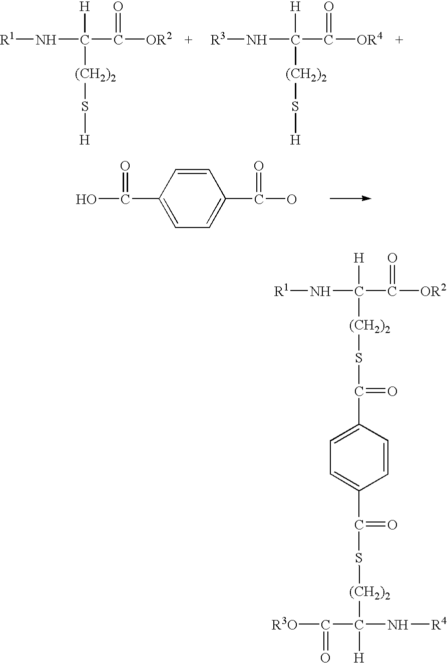

wherein R¹ and R² represent the remainder of a first water soluble protein molecule and R³ and R⁴ represents the remainder of a second water soluble protein molecule, preferably different α-keratin molecules.

The multi-ester or anhydride typically is at a concentration of up to about 20 weight percent relative to keratin, preferably between 5 and 10 weight percent relative to keratin, most preferably about 10 weight %. The pH is less than about 7, preferably from about 5 to about 7. The temperature is from about 0 to about 100° C., preferably about 60° C. The exposure is continued for a time period of from about 1 hour to about 72 hours, most preferably about 24 hours. Mineral acid catalysts, such as hydrochloric acid, typically can be employed.

Addition to unsaturated hydrocarbon

Addition reactions, such as free radical addition to an unsaturated hydrocarbon represents another potential avenue to transformation of the thiol group. A variety of allyl derivatives, for example, can be used to modify the thiol in the presence of an appropriate catalyst or free radical initiator. Many free radical initiators exist that are conveniently activated by heat or light. The free radical addition reaction scenario is shown in the following formula:

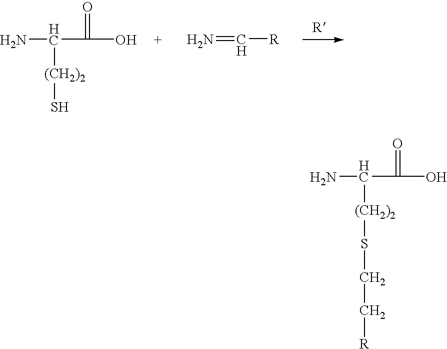

Addition of isocyanate to hydroxyl groups

In another embodiment, a diisocyanate is reacted with hydroxyl groups in the keratin, such as those contained in serine. The reaction is shown below:

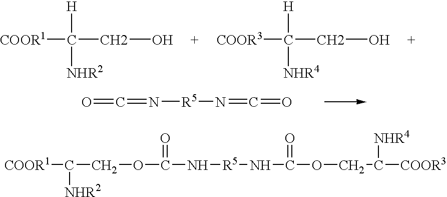

wherein R¹ and R² represent the remainder of one water soluble protein molecule, and R³ and R⁴ represent the remainder of a second water soluble protein molecule preferably a different α-keratin molecule. R⁵ may be a variety of organic moieties effective to produce hydrogels having the desired properties. In a preferred embodiment, R⁵ is selected from the group consisting of aryl groups, including benzyl groups, and alkyl and allyl groups having from about 1 to about 8 carbon atoms, and polyalkylethers containing from about 1 to 50 repeat groups. In a preferred embodiment, R⁵ is an alkyl group having 6 carbon atoms.

A similar reaction occurs with arginine:

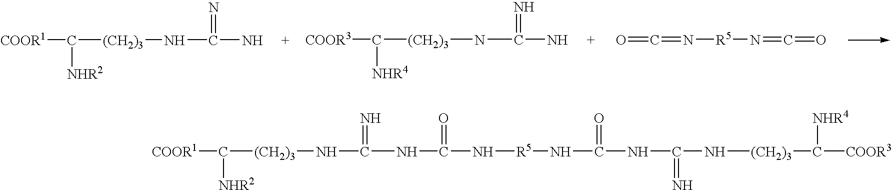

A similar reaction occurs with cysteine, as follows:

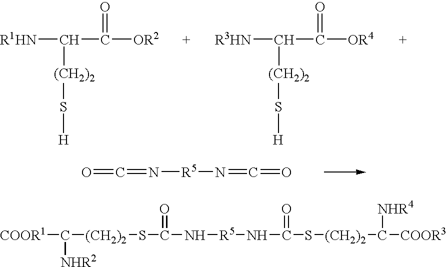

In order to perform these reactions, the crosslinking agent is preferably dissolved in an anhydrous solvent, such as methanol, ethanol, isopropyl alcohol, dimethylsulfoxide, acetone, or tetrahydrofuran. The concentration of this solution is such that the appropriate amount of crosslinking agent is added as the hydrogel is formed. For example, to form a hydrogel with 10 wt. % crosslinking agent and 5 wt. % HMWK solids, 1 gram of a solution containing 0.1 grams of crosslinking agent dissolved in the anhydrous solvent is added to 1.0 gram of HMWK powder dissolved in 18 gm of water. The solution and powder are thoroughly mixed and a hydrogel spontaneously forms. This hydrogel is allowed to react under the time and temperature conditions described previously to effect crosslinking. Crosslinking reactions can be accelerated by incubating this gel in a closed atmosphere at a temperature of from about 25° C. to about 80° C., preferably 37° C.

Persons of ordinary skill in the art will recognize that many of the crosslinking agents described herein will react with a variety of amino acid residues having pendant groups comprising a reactive nitrogen atom, sulfur atom, or oxygen atom. Hence, one end of a diepoxide may react with a cysteine residue while the other end of the diepoxide reacts with an arginine residue, as follows:

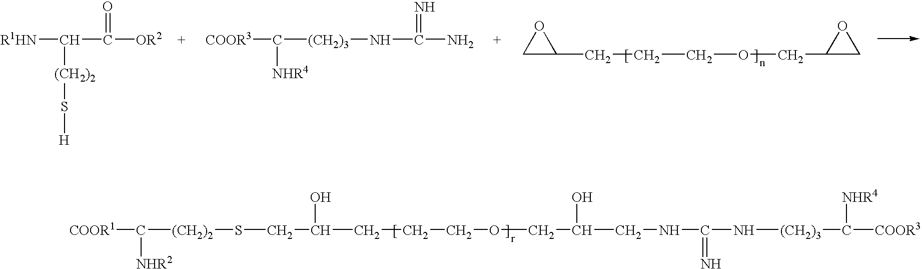

The identity of amino acid residues linked by the crosslinking agent is not as important as the requirement that a sufficient quantity of crosslinking between water soluble proteins occurs to produce a network and preferably a hydrogel having desired properties.

HETEROGENEOUS NETWORKS OR FILMS

In an alternative tissue defect dressing, the proteinaceous salve comprises a heterogeneous crosslinked proteinaceous network. As used herein, the term “heterogeneous” refers to a proteinaceous network or film, preferably comprising protein molecules having a relatively high molecular weight of from about 50 to about 85 kDa, or derivatives therefrom. The protein molecules are interlinked by a non-proteinaceous crosslinking material.

As with hydrogels, a variety of proteins can be used to form heterogeneous network structures, preferably elastomeric films. Examples of suitable naturally occurring proteins include, but are not necessarily limited to keratin, collagen, and elastin. The proteins may be natural, synthetic, or recombinant. Preferred proteins are relatively high in cysteine content. Most preferred proteins are keratin proteins, even more preferably a-keratin proteins, also sometimes called high molecular weight keratins (HMWK's).

Disulfide bond scission and keratin extraction

The proteins, preferably α-keratins, may be processed and/or isolated in any manner that renders them sufficiently soluble in the reaction media for crosslinking reaction(s) to occur. A number of the reactions described below call for an anhydrous solvent. Persons of ordinary skill in the art will recognize that anhydrous solvents include a large number of solvents, including, but not necessarily limited to 1,2,-dimethoxyethane, dimethylformamide, dimethylsulfoxide (DMSO), N-methyl pyrrolidone, and others. Generally, the reactions require the presence of at least some water.

Oxidation/Reduction of Cystine Residues

In a preferred embodiment, which uses keratins as a source material (e.g. human hair), the hair is oxidized by a suitable oxidizing agent. Suitable oxidizing agents include, but are not necessarily limited to hydrogen peroxide, peracetic acid, percarbonates, persulfates, chlorine dioxide, sodium and calcium peroxides, perborates, and hypochlorite. The oxidants are used at a concentration of up to about 35%, preferably at from about 0.1% to about 10%. The oxidation preferably occurs at reflux temperatures.

In a preferred embodiment, the hair is treated with hydrogen peroxide (H₂O₂), at from about 0.1% to about 10%, most preferably 1%, in order to disrupt the cuticle and swell the keratin source material. This process also converts some fraction of the cystine residues into sulfonic acid groups. The amount of oxidation may be controlled by varying the time of oxidation, preferably from about 0 hours to about 4 hours, while retaining the other conditions of the oxidation reaction constant. These conditions include concentration and type of oxidant, temperature, and ratio of extracting media to keratin source material. After the reaction is complete, the oxidized hair is filtered and rinsed, preferably with deionized water. The filtrate is discarded and the hair allowed to dry.

Where other conditions of oxidation are maintained constant, the conversion rate of cystine to sulfonic acid residues is roughly proportional to the amount of time used for the oxidation. Residual cystines in the resulting oxidized keratin solids are converted to other sulfur-containing moieties using reductive techniques. Preferably, the disulfide-bridged cystine group is converted to a thiol group, which has utility of it's own, or can be modified using a variety of chemical techniques.

Reaction with a Reducing Agent

If oxidized, the oxidized hair preferably is treated with a reducing agent. Treatment of oxidized keratin proteins with reducing agents facilitates the formation of cysteine from cystine, but tends to leave the previously oxidized groups unaltered. Suitable reducing agents include, but are not necessarily limited to thioglycolic acid and salts thereof, mercaptoethanol, dithiothreitol, thioglycerol, thiolactic acid, glutathione, cysteine, sodium sulfide, and sodium hydrosulfide. Preferred reducing agents are thioglycolic acid and mercaptoethanol, most preferably thioglycolic acid.

In order to treat the oxidized hair with the reducing agent, the previously oxidized hair is suspended in the reducing agent typically at a concentration of up to about 10N, preferably from about 0.1N and 1N; at a pH greater than about 7, preferably equal to or greater than 9, most preferably 9; a temperature of from about 25 to about 80° C., preferably about 60° C., preferably for a time period of from about 1 to about 72, most preferably about 24 hours. The reaction occurs under an inert atmosphere, preferably nitrogen. The liquid fraction is separated from any remaining solids using known means, including but not necessarily limited to filtration, or cannulation and/or centrifugation, preferably under inert atmosphere. A preferred method of separation is filtration. Once the solids are removed, the soluble keratin proteins are isolated from the solution by addition of a water-miscible non-solvent, or by spray drying. Water-miscible non-solvents include, but are not necessarily limited to ethanol, methanol, isopropyl alcohol, tetrahydrofuran, acetone, dioxane, and the like, again under inert atmosphere. A preferred non-solvent is ethanol. The precipitate is separated from the non-solvent using known means, preferably by filtration and rinsing using additional aliquots of the non-solvent. The resulting keratin proteins are dried using known techniques, preferably overnight under vacuum at room temperature. This process results in the keratins having both sulfonic acid groups and thiol groups.

Thiols possess reactivities similar to alcohols, and can be used to perform a multitude of known organic chemical reactions, such as those described in McMurry, J., Organic Chemistry, Brooks/Cole Publishing Co., Monterey, Calif. (1984); Scudder, P. H., Electron Flow in Organic Chemistry, John Wiley & Sons, New York, N.Y. (1992); Stowell, J. C., Intermediate Organic Chemistry, John Wiley & Sons, New York, N.Y. (1994), incorporated herein by reference. The ratio of sulfonic acid to thiol is primarily controlled by the quantity of primary reactive sites remaining after oxidation. Of course, the rate of reduction will also be affected by reagent concentration(s), reaction temperature(s), and exposure time(s).

Reductive/reductive extraction

Reductive chemistries also are known for disulfide bond scission in keratins: See Wardell, J. L., “Preparation of Thiols” in The Chemistry of the Thiol Group, Patai, S. (Editor), pp. 163-353, John Wiley & Sons, New York, N.Y. (1974), incorporated herein by reference. HMWK's may be extracted from hair using at least two reductive extractions, as described in Crewther, W. G., Fraser, R. D. B., Lennox, F. G., and Lindley, H., “The Chemistry of Keratins” in Advances in Protein Chemistry, Anfinsen, C. B., Jr., Anson, M. L., Edsall, J. T., and Richards, F. M. (Editors), Academic Press, New York, pp. 191-346 (1965), incorporated herein by reference.

Briefly, in a first reductive extraction, the hair is treated with a first reducing agent under first conditions effective to selectively extract matrix keratins, producing a first solution comprising soluble reduced matrix keratins and remaining hair solids. The remaining hair solids and the first solution are separated, and the remaining hair solids are exposed to a second extraction solution under second conditions effective to solubilize α-keratins, producing a second solution comprising soluble reduced α-keratins and solid cuticle. Once the second extraction is complete, the remaining solids essentially are the empty, intact cuticle.

The liquid fraction is separated from the solid cuticle using known means, including but not necessarily limited to filtration, or cannulation and/or centrifugation, preferably under inert atmosphere. A preferred method of separation is filtration. Once the solids are removed, the soluble keratin proteins are isolated from the solution by addition of a water-miscible non-solvent, or by spray drying. Water-miscible non-solvents include, but are not necessarily limited to ethanol, methanol, isopropyl alcohol, tetrahydrofuran, acetone, dioxane, and the like, again under inert atmosphere. A preferred non-solvent is ethanol. The precipitate is separated from the non-solvent using known means, preferably by filtration and rinsing using additional aliquots of the non-solvent. The resulting keratin proteins are dried using known techniques, preferably overnight under vacuum at room temperature. The dried keratin proteins are ground into a powder, sometimes referred to as “HMWK powder.”

Network formation

In a preferred embodiment, which uses HMWK proteins, the HMWK proteins are dissolved in an aqueous solvent. Preferably, about 2 g of HMWK powder is mixed in water containing a suitable base, and the mixture is stirred and heated to a temperature effective to dissolve the keratin, typically not more than 60° C. The pH of the solution is maintained at about 9 to 11 using a suitable base. Suitable bases include, but are not necessarily limited to ammonium hydroxide, sodium hydroxide, and potassium hydroxide, preferably ammonium hydroxide. At least about 5 wt. %, preferably about 10 wt. %, relative to the keratin, of a multifunctional crosslinking agent is added to the mixture, forming a network precursor solution. Depending upon the crosslinking agent, a catalyst or promoter may be added. The network precursor solution is distributed over an appropriate surface or mold, preferably to a thickness of from about 1 to about 10 mm, and cured by exposure to suitable energy, such as a heat lamp, an autoclave, a microwave, or a UV lamp. In a preferred embodiment, the solutions are placed under a heat lamp effective to produce a temperature of at least about 60° C. for from about 30 to 300 minutes.

Crosslinking reactions

Crosslinking of the proteins and network formation occurs, generally, when a non-protein reactant which is at least difunctional, or has at least two reactive groups, is used to crosslink between reactive pendant groups on two different keratin molecules. The non-protein reactant creates a bridge between keratin molecules, and thus produces a three-dimensional network. The chemistries described above for the preparation of hydrogels also are useful for the preparation of heterogeneous films. A preferred embodiment for the preparation of heterogeneous films involves the use of diepoxides or oxiranes, and is described above in relation to hydrogels under the title “Addition of Amine Groups.” In addition, the following chemistries also are useful to prepare hydrogels:

Production of thioether

A preferred reductive modification is the formation of a thiolate anion, followed by nucleophilic substitution employing an appropriate leaving group, yielding a thioether, preferably an alkoxy functional thioether (or a thioester). A preferred alkoxy functional thioether is an epoxy-functional thioether. The general reaction is shown below:

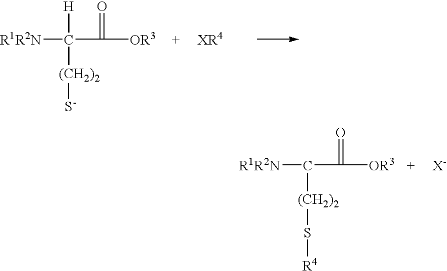

wherein R¹ and R² comprise entities selected from the group consisting of hydrogen and the remainder of the N-terminal portion of the protein molecule; R³ comprises the remainder of the carboxy-terminal portion of the protein molecule; and, R⁴ is a group adapted to form a thioether, preferably an alkoxy functional thioether. Suitable R⁴ groups comprise a “substitution end,” which bonds with the sulfur and a “reactive end” which reacts with the crosslinking agent. Suitable substitution ends include, but are not necessarily limited to unsubstituted and halo-substituted alkyl groups and alkylene groups having from about 1 to about 8 carbon atoms, including resonance hybrids, such as allyl groups, and unsubstituted and halo-substituted aryl groups. Suitable reactive ends include, but are not necessarily limited to acyl groups, and polyalkylethers containing from about 1 to 50 repeat groups, isocyanate groups, silane groups, and silicone groups. Preferred reactive ends include, but are not necessarily limited to carboxyl groups, isocyanate groups, and alkoxide groups. A most preferred reactive end is an epoxide group. In the foregoing formula, X may be any appropriate leaving group. Suitable leaving groups include, but are not necessarily limited to halide groups, tosylate groups, acetate groups, hydroxyl groups, alkoxy groups, and amine groups. Preferred XR⁴ groups are halides, most preferably chlorine. In a most preferred embodiment, XR⁴ is epichlorohydrin.

The thiolate anion can be generated from thiol, or more directly from the water soluble peptide feedstock, preferably a keratin feedstock, by reaction with a reactive nucleophile. Suitable nucleophiles include alkyl and aryl functional sulfide salts, sulfonates, isocyanates, thiocyanates, halides, hydrosulfide, hydroxide, alkoxides, azides, and acetates preferably alkyl and aryl sulfide salts, hydrosulfide, hydroxide, alkoxides, azides, and acetates.

The reaction where RX is epichlorohydrin is shown below:

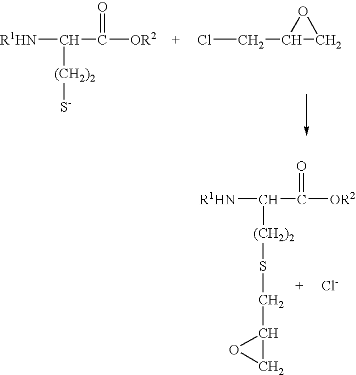

wherein R¹ and R² are the remainder of the water soluble peptide molecule of which cysteine is a part.

In order to form the foregoing epoxide functionalized water soluble peptides, preferably water soluble keratins, a water soluble keratin source material is first produced, preferably as described above. The water soluble keratins are then exposed to a solution of “RX”, preferably epichlorohydrin, in aqueous solution at a pH of from about 9 to about 11. The RX is typically at a concentration of up to about 20 mole percent relative to keratin, preferably from about 5 to 10 mole percent relative to keratin, most preferably about 10 mole. %. The pH is greater than about 7, preferably greater than 9. The temperature is from about 20 to about 100° C., preferably about 60° C. The reaction continues for a time period of from about 1 to about 72 hours, most preferably about 24 hours. The result is epoxidized thiol groups.

The resulting epoxy functional thioether is reacted with a reactive pendant group on a second water soluble keratin peptide, including but not necessarily limited to a thiol group and a reactive nitrogen-containing group, such as an amine group. The following is an illustration of a crosslinking reaction between an epoxy functional thioether on one water soluble peptide and an arginine residue on another water soluble peptide:

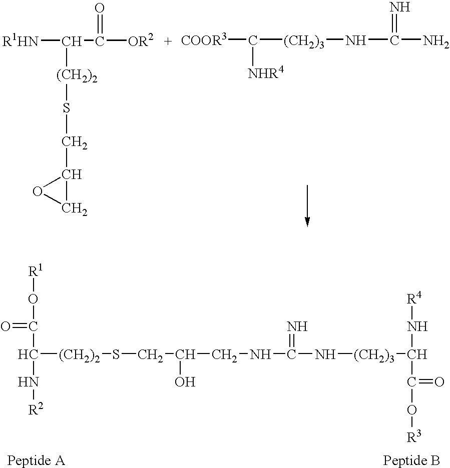

R¹ and R² are the remainder of the water soluble peptide “A” bearing the epoxy functionalized cysteine, and R³ and R⁴ are the remainder of the water soluble peptide molecule B, containing the arginine residue. Although it is theoretically possible for water soluble peptide molecule A and B to be the same molecule, it is preferred for peptides A and B to be different molecules, preferably different water soluble α-keratin molecules.

Multifunctional silicone-based material

In another embodiment of a heterogeneous crosslinked film, the crosslinking agent is a multifunctional silicone-based material. Silicones are a family of biocompatible materials that have been used in a myriad of medical applications. Silicone gel sheeting, a form of lightly crosslinked silicone polymer, promotes wound healing and lessens the degree of hypertrophic scar formation. The technology of silicone chemistry is varied and useful, particularly with respect to elastomer formation, as many crosslinking modalities have been developed. Thomas, D. R., “Cross-linking of Polydimethylsiloxanes”, in Siloxane Polymers, Clarson, S. J. and Semlyen, J. A. (Editors), PTR Prentice Hall, N.J., pp. 567-615 (1993), incorporated herein by reference. Many of these crosslinking chemistries can be adapted for use in other systems such that copolymer and interpenetrating networks comprising at least some silicone have been produced. The beneficial wound healing attributes of silicone biomaterials, combined with their flexible chemistry, makes them ideal candidates for crosslinking keratin-based biomaterials.

Silicones are bioinert and resilient in biological systems. A bioinert crosslinking agent has the advantage of maintaining the biological stealth of the system of which it is a part. The combination of keratins with silicone-based crosslinking agents combines the wound healing efficacy of both biomaterials without compromising the inherent biocompatibility of keratins.

Suitable silicone cross-linking agents, or polysiloxanes, are molecules having recurring Si-O linkages:

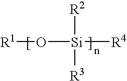

wherein n is from about 1 to about 50, and R¹, R², R³, and R⁴ can be a large variety of groups, wherein at least two of R¹, R², R³, and R⁴ comprise a “reactive functionality,” defined as a functionality that is reactive toward reactive pendant groups on the protein molecules to be interlinked. Suitable reactive functionalities comprise one or more reactive moieties selected from the group consisting of reactive unsaturated carbon-carbon bonds, reactive oxygens, reactive nitrogens, reactive sulfurs, and reactive halogens. Preferred reactive functionalities include, but are not necessarily limited to reactive unsaturated carbon-carbon bonds, hydrido groups, hydroxyl groups, alkylamine groups, alkylmercapto groups, alkoxy groups, trifluoroalkyl groups, wherein the alkyl moiety comprises from about 1 to about 6 carbon atoms. A preferred trifluoroalkyl group is a trifluoropropyl group; a preferred alkoxy group is an epoxy group, and a preferred unsaturated carbon-carbon bond is a vinyl group.

Examples of suitable R¹, R², R³, and R⁴ groups, some of which are reactive functionalities and some of which are not, include, but are not necessarily limited to hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups (sometimes referred to herein as “alkyl ethers”) comprising one or more alkyl moieties having a total of from about 1 to about 6 carbon atoms, hydrido groups, and hydroxyl groups. Preferred heteroalkyl groups include, but are not necessarily limited to acetoxy groups, silane groups optionally comprising one or more alkyl substituent having a total of from about 1 to about 6 carbon atoms, and combinations thereof. Preferred alkoxy groups include, but are not necessarily limited to epoxy groups.

Preferably, R¹ and R⁴ are moieties comprising reactive functionalities which are adapted to react with complementary functional groups on the protein molecules to be interlinked, preferably α-keratin molecules. In a preferred embodiment, R¹ and R⁴ independently are selected from the group consisting of hydrogen, linear, branched or cyclic alkyl groups having from about 1 to about 6 carbon atoms, alkenyl groups having from about 2 to about 6 carbon atoms, hydrido groups, alkoxy groups comprising one or more alkyl groups having a total of from about 1 to about 6 carbon atoms, hydroxy groups, alkylamine groups, alkylmercapto groups, acrylate groups, methacrylate groups, halo groups, acetoxy groups, and epoxy groups. In a more preferred embodiment, both R¹ and R⁴ comprise a moiety selected from the group consisting of vinyl groups and epoxy groups. Most preferably, R¹ and R⁴ comprise the same moiety selected from the group consisting of vinyl groups and epoxy groups; and

In a preferred embodiment, R² and R³ independently are selected from the group consisting of hydrogen, cycloalkyl groups, vinyl groups, hydrido groups, trifluoroalkyl groups, phenyl groups, alkyl groups, alkoxy groups, alkylmercapto groups, and alkylamine groups; provided that, when one of R² or R³ is a vinyl group, the other of R² or R³ is a group other than a hydrido group; and, when one of R² or R³ is a hydrido group, the other of R² or R³ is a group other than a vinyl group. In an even more preferred embodiment, R² and R³ preferably are relatively inert groups. Most preferably at least one of R² and R³ is an alkyl group, more preferably a methyl group.

Commercially available silicone products include, but are not necessarily limited to vinyl functional, alkoxy functional (preferably epoxy functional), alkylamine functional, hydroxyl functional, and alkylmercapto functional polysiloxy polymers and copolymers, which are available, for example, from Gelest, Inc., Tullytown, Pa., or may be made using known methods, such as those described in Thomas, D. R., “Cross-linking of Polydimethylsiloxanes”, in Siloxane Polymers, Clarson, S. J. and Semlyen, J. A. (Editors), PTR Prentice Hall, N.J., pp. 567-615 (1993), incorporated herein by reference. Most preferred commercially available vinyl functional products are generally available in molecular weights ranging from about 363 to about 5,500, with preferred molecular weights being from about 500 to about 3500.

A preferred crosslinking agent is the epoxycyclohexyl copolymer discussed in more detail below. More preferred is epoxypropoxypropyl-terminated silicones discussed in more detail below. Most preferred crosslinking agents are vinyl-terminated silicones. These crosslinking agents may be obtained, for example from Gelest, Inc., Tullytown, Pa., or prepared using known procedures.

(Epoxycyclohexylethyl)methylsiloxane-dimethylsiloxane copolymer generally is available in molecular weights ranging from 500 to 50,000, with preferred molecular weights being from about 650 to about 3500. (Epoxycyclohexylethyl)methylsiloxane-dimethyl siloxane copolymer has the following general structure:

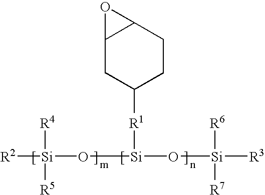

wherein m and n add to a total of from about 5 to about 50; R² and R³ may be any of the groups listed as end groups R¹ and R⁴ in the general formula for the silicone cross-linking agents given at the beginning of this section, and R¹, and R⁴-R⁷ may be any of the substituents listed as the Si-substituents R² and R³ in the general formula for the silicone cross-linking agents given at the beginning of this section. Preferably, R¹ and R⁴-R⁷ are selected from the group consisting of alkyl groups having from about 1 to about 6 carbon atoms. R¹ preferably is a methyl group; R⁴-R⁷ preferably are methyl groups. A preferred (epoxycyclohexylethyl)methylsiloxane-dimethylsiloxane copolymer, which is commercially available from Gelest, has the following general structure:

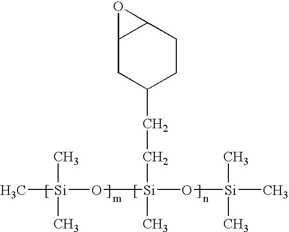

Preferred silicone-based crosslinking agents react with reactive groups on the protein molecules to produce biocompatible byproducts, preferably hydrogen, water, carbon dioxide, and/or any other biocompatible byproduct that is readily metabolized or excreted, removed from the network, or at least is not toxic to the human body. Suitable silicone-based crosslinking agents have either two or more of the same reactive functionalities, or two or more different reactive functionalities. Preferred silicone-based crosslinking agents have two or more of the same functional group.

Network properties

As seen, a three dimensional keratin-based network can be formed using a variety of chemistries. Preferably, the “dissolution rate” of such a network is controllable by controlling the crosslink density of the film and the level and type of functionality, particularly the functionality adjacent to the crosslink site. For example, the use of a crosslinking agent having one of the following characteristics reduces the dissolution rate of the resulting network: a crosslinking agent which forms S-C bonds, as opposed to more hydrolyzable bonds, such as ester bonds; a crosslinking agent which introduces substantial steric hindrance at the crosslink site; a crosslinking agent which is hydrophobic. The “dissolution rate” of the resulting network or film is measured by determining how long the film resists hydrolysis upon exposure to an aqueous buffer having a pH of about 7. A desirable “dissolution rate” will depend upon the application in which the film or hydrogel is to be used.

The invention will be better understood with reference to the following Examples, which are illustrative only:

### EXAMPLE 1

800 mL of 0.8M thioglycolic acid (TGA) at pH 10.2, adjusted by addition of 52.58 grams of potassium hydroxide (KOH), was added to a 1 L glass reactor containing 40 grams of human hair obtained from a local barber shop. The hair had been washed with an aqueous solution of mild detergent, rinsed, and air dried prior to use. The mixture was stirred gently at room temperature (ca. 25° C.) under positive nitrogen pressure for 18 hours.

The reaction mixture was filtered and the extract titrated to pH 7 by addition of hydrochloric acid (HClo). The neutralized solution was added dropwise to a 10-fold excess of absolute ethanol, while stirring, to promote the formation of a precipitate. The precipitate was filtered and dried under vacuum.

The remaining reduced hair was further processed by first rinsing it free of residual reductant using copious amounts of deionized water, upon which the hair swelled to nearly twice its initial volume. The rinsed hair was placed in a 2 L reactor to which was added 400 mL of 7M urea solution. The reaction was stirred at room temperature under positive nitrogen pressure for 24 hours.

100 mL of the reaction mixture was centrifuged and filtered. The pH of the filtered extract was titrated to pH 7 with the addition of HCl. The neutralized solution was added dropwise to 900 mL of absolute ethanol to affect precipitation. The solid keratins were isolated by filtration and hydrated with 18 MΩ water, upon which, a thick hydrogel formed.

### EXAMPLE 2

800 mL of 1M mercaptoethanol at pH 10.2, adjusted by addition of ca. 24 grams of KOH, was added to a 1 L glass reactor containing 40 grams of human hair obtained from a local barber shop. The hair had been washed with an aqueous solution of mild detergent, rinsed, and air dried prior to use. The mixture was stirred gently at room temperature under positive nitrogen pressure for 18 hours. After extraction, the mixture was filtered and the liquid discarded.

The remaining reduced hair was further processed by first rinsing it free of residual reductant using copious amounts of deionized water, upon which, the hair swelled to nearly twice its initial volume. The rinsed hair was placed in a 2 L reactor to which was added 400 mL of 7M urea solution. The reaction was stirred at room temperature under positive nitrogen pressure for 24 hours.

The reaction mixture was centrifuged and filtered. The pH of the filtered extract was titrated to 7 with the addition of HCl. The neutralized solution was added dropwise to 4,500 mL of absolute ethanol to affect precipitation. The solid keratins were isolated by filtration and dried overnight under vacuum. The dried keratin powder was ground to a medium consistency with a mortar and pestle.

1.0 grams of a 0.01M sodium hydroxide (NaOH) solution was added to a vial containing 0.1 grams of the keratin powder from Example 2, upon which, the mixture formed a thick hydrogel. Another 2.0 grams of 0.01M sodium hydroxide solution was added (3.0 grams total) which reduced the viscosity of the hydrogel only slightly. This process was repeated with 0.001M and 0.0001M NaOH solutions with similar results.

0.5 grams of a 30% ammonium hydroxide (NH₄OH) solution was added to a vial containing 0.1 grams of the sample from Example 2, upon which, the mixture formed a thick hydrogel. Subsequent additions of 0.5, 1.0, and 0.5 grams of NH₄OH did not appear to reduce the viscosity of the gel. Addition of a final 0.5 grams of NH₄OH (3.0 grams total) reduced the viscosity only slightly.

### EXAMPLE 3

800 mL of 1M mercaptoethanol at pH 10.2, adjusted by addition of ca. 25 grams of KOH, was added to a 1 L glass reactor containing 40 grams of human hair obtained from a local barber shop. The hair had been washed with an aqueous solution of mild detergent, rinsed, and air dried prior to use. The mixture was stirred gently at room temperature under positive nitrogen pressure for 18 hours.

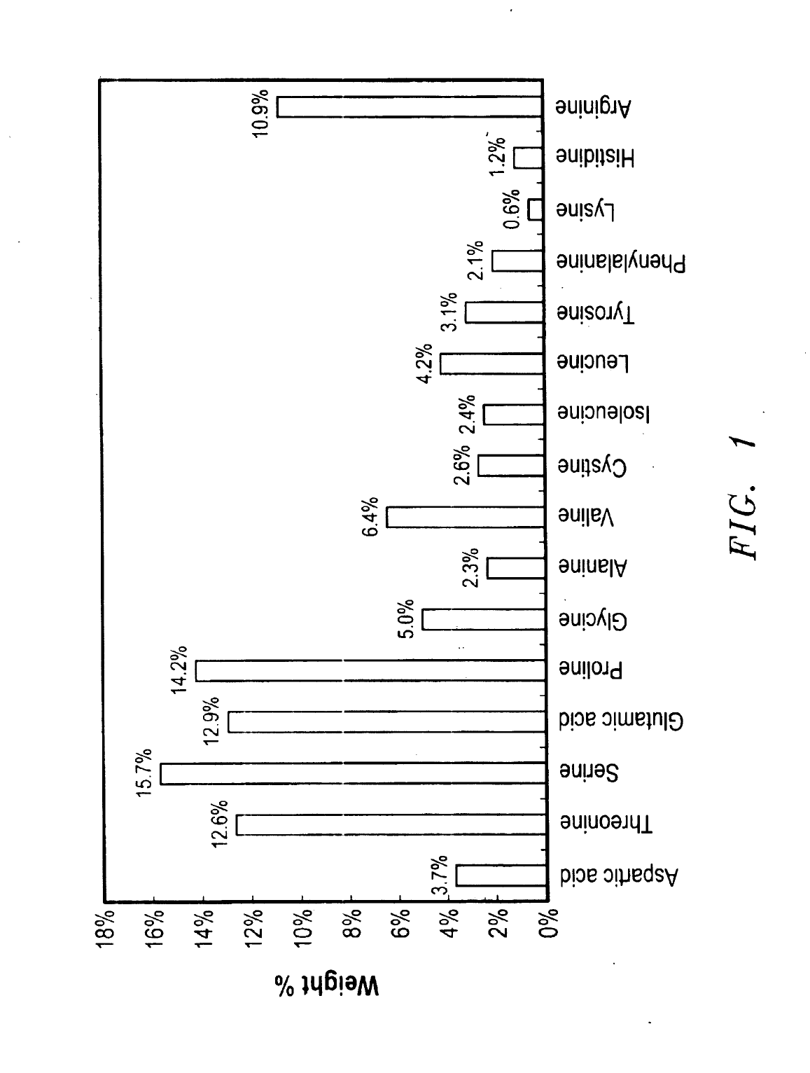

The reaction mixture was filtered and the extract titrated to pH 7 by addition of HCl. The neutralized solution was added dropwise to a 10-fold excess of absolute ethanol while stirring to promote the formation of a precipitate. The precipitate was filtered and dried under vacuum. Yield of keratin solids was 13.37 grams (33.4%). An amino acid analysis of this sample was conducted by acid hydrolysis and dissolution of the solid, followed by high pressure liquid chromatography (HPLC) of the solution. For cysteine residues, an analysis was performed on a separate sample by first oxidizing the solid keratins with performic acid, followed by acid digestion and HPLC analysis. The results of these analyses are summarized in the graph in FIG. 1.

The remaining reduced hair was further processed by first rinsing it of residual reductant using copious amounts of deionized water, upon which, the hair swelled to nearly twice its initial volume. The rinsed hair was placed in a 1 L reactor to which was added 400 mL of 7M urea solution. The reaction was stirred at room temperature under positive nitrogen pressure for 24 hours.

The reaction mixture was centrifuged and filtered. The pH of the filtered extract was titrated to 7 with the addition of HCl. The neutralized solution was added dropwise to 4,000 mL of absolute ethanol to affect precipitation. The solid keratins were isolated by filtration and dried overnight under vacuum. The dried keratin powder was ground to a medium consistency with a mortar and pestle. The yield of keratin solids was 1.66 grams (sample no. 4-AKR-1 12-2B; 4%).

Three sets of duplicate hydrogels containing 0.1 gram each of the keratin solids and 1.0, 2.0, and 3.0 grams of 30% NH₄OH solution were prepared in glass vials. The weight percent solids in each of these duplicate gels were 9.1, 4.8, and 3.2, respectively. The headspace of one of each of the duplicates was filled with pH 7.2 phosphate buffered saline and the vial capped and sealed with tape. These samples, as well as the set containing no buffer solution (also capped and sealed with tape) were placed in an incubator held at 37° C. (body temperature) and monitored periodically by visual observation.

After 4 days in the incubator, the 2.0-gram gel in saline appear to begin disintegrating into the aqueous layer. There were no visible signs of any changes in the other hydrogels. After 16 days, all of the gels appeared intact, however, the buffer solutions displayed a slight brown discoloration. After 21 days, the gels were removed from the incubator. Other than a slight discoloration of the buffer solution, and a small amount of volume shrinkage in the buffered samples, no changes in any of the hydrogels appeared to have occurred.

### EXAMPLE 4

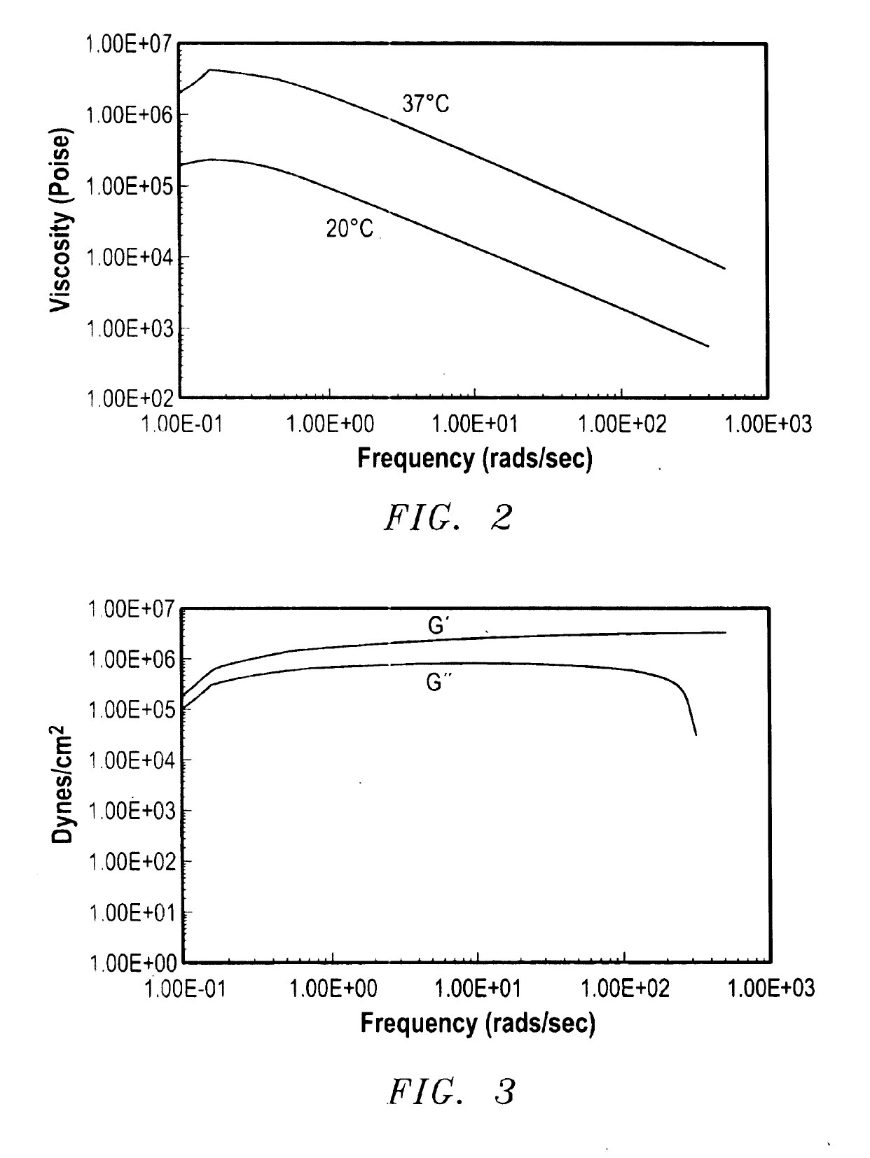

1.0 grams of a 30% ammonium hydroxide (NH₄OH) solution was added to a vial containing 0.1 grams of human hair from Example 3. The mixture formed a thick hydrogel. The viscosity of this gel was analyzed using a cone and plate rheometer. The results, shown in FIGS. 2 and 3 suggest that the hydrogel is a classical non-Newtonian fluid.

In a “Newtonian fluid,” the coefficient of viscosity at a given temperature and pressure is a constant for that fluid and independent of the rate of shear or velocity gradient. Non-Newtonian fluids consist of two or more phases present at the same time, and the coefficient of viscosity is not constant, but is a function of the rate at which the fluid is sheared as well as of the relative concentration of the phases. Non-Newtonian fluids frequently exhibit plastic flow, in which the flowing behavior of the material occurs after applied stress reaches a critical value or yield point (YP).

The fact that the hydrogel formed was a non-Newtonian fluid served to indicate that the bonding, such as pseudo-bonding or polymer entanglements had occurred within the gel.

### EXAMPLE 5

3,500 mL of 1.0M thioglycolic acid (TGA) at pH 10.2, adjusted by addition of 312.5 grams of potassium hydroxide (KOH), was added to a 4 L glass reactor containing 175 grams of human hair obtained from a local barber shop. The hair had been washed with an aqueous solution of mild detergent, rinsed, and air dried prior to use. The mixture was stirred gently at room temperature (ca. 25° C.) under positive nitrogen pressure for 18 hours.

The reaction mixture was sieved and the reduced hair was further processed by first rinsing it free of residual reductant using copious amounts of deionized water, upon which the hair swelled to nearly twice its initial volume. The rinsed hair was placed into two separate glass reactors to which was added 7M urea solution. These extractions were stirred at room temperature under positive nitrogen pressure for 24 hours.

The extracted hair was removed from solution by passing through a sieve. The liquid fraction was centrifuged and filter, then neutralized to pH 7 with the addition of HCl (3.7 mL). The liquid was added dropwise to a 10-fold excess of ethanol to effect precipitation of the HMWKs. The precipitate was isolated by filtration, washed with several aliquots of fresh ethanol, then dried overnight under vacuum. The resulting solid HMWKs were ground to a fine powder using a mortar and pestle.

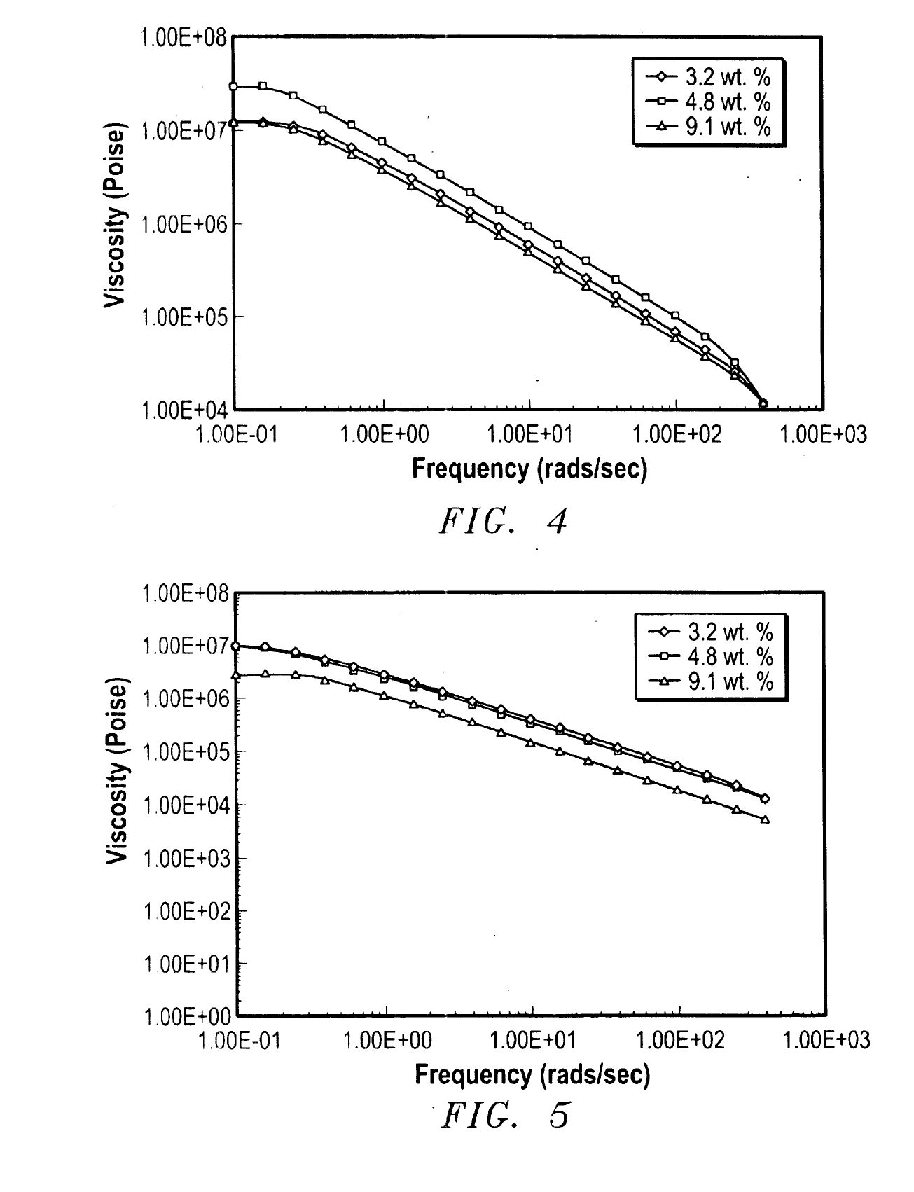

Nine-100 milligram samples of the powder were placed in glass vials. To each set of three vials was added 1.0, 2.0, and 3.0 grams of deionized water, respectively. The contents of each vial were mixed and allowed to gel. The resulting triplicate set of hydrogels contained 9.1, 4.8, and 3.2 weight % keratins, respectively. To one of each triplicate hydrogel was added enough pH 7.2 phosphate buffered saline solution to fill the headspace of the vial. These samples, as well as identical samples not containing buffer solution, were placed in an incubator held at a constant temperature of 37° C. The third sample of each triplicate was incubated at 37° C. for 24 hours, then analyzed using a cone and plate rheometer. The rheometer was used to determine the shear dependant viscosity at 37° C. of these “un-crosslinked” hydrogels. As used herein, “un-crosslinked” describes hydrogels that have not been reacted with a multifunctional crosslinker per se; such hydrogels will have virtual and pseudo crosslinks present. FIG. 4 shows the results for the 9.1, 4.8, and 3.2 wt. % hydrogels.

Similarly, nine-100 milligram samples of the powder were placed in glass vials. To triplicate samples was added 1.0, 2.0, and 3.0 grams of solutions containing 1.0, 0.5, and 0.33 wt. % of the crosslinker, DER 332 resin, respectively. This was done in order to deliver 0.01 grams (10 wt. % relative to keratin) of dissolved crosslinker to each vial of keratin powder.

To one of each triplicate hydrogel was added enough pH 7.2 phosphate buffered saline solution to fill the headspace of the vial. These samples, as well as identical samples not containing buffer solution, were placed in an incubator held at a constant temperature of 37° C. The third sample of each triplicate was incubated at 37° C. for 24 hours, then analyzed using a cone and plate rheometer. The rheometer was used to determine the shear dependant viscosity at 37° C. of these crosslinked hydrogels. FIG. 5 show the results for the 9.1, 4.8, and 3.2 wt. % hydrogels containing DER 332 resin crosslinks.

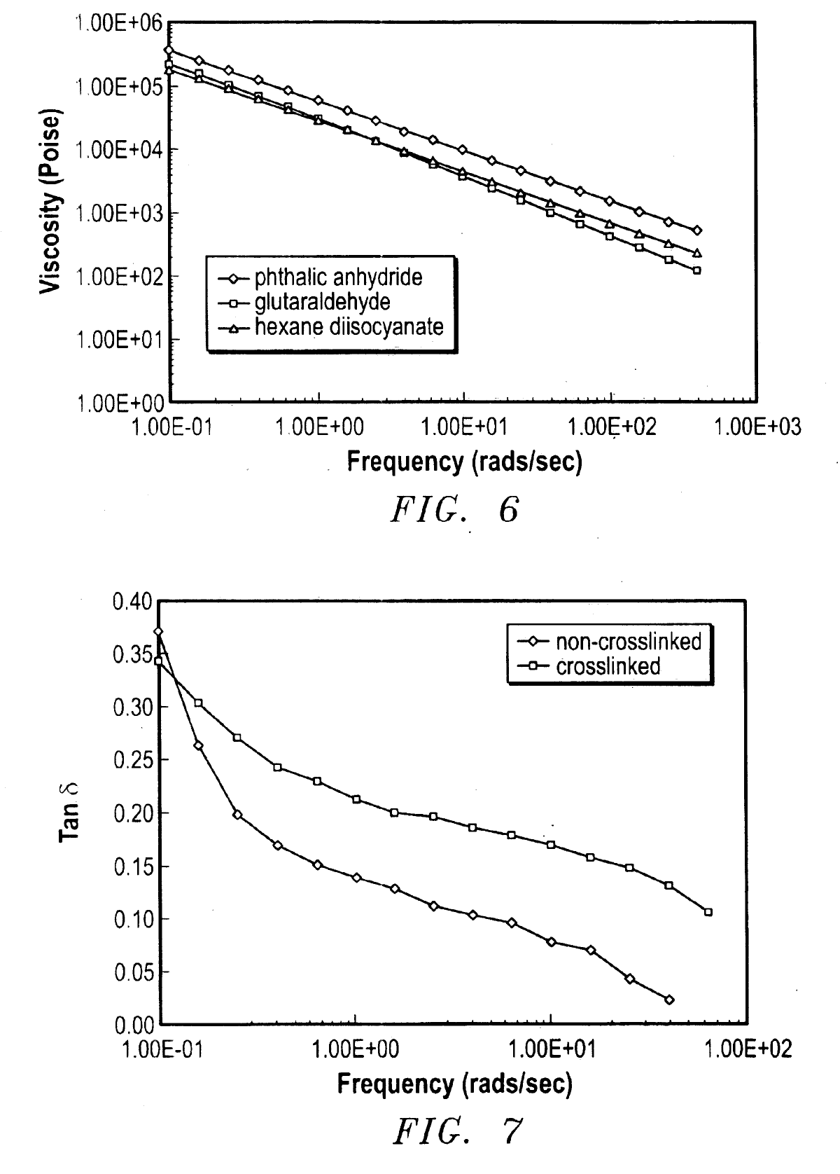

This process was repeated using phthalic anhydride and glutaraldehyde as crosslinkers. Hexane diisocyanate was also used as a crosslinker, but was added directly to the hydrogel as it was forming. The viscosities of the 4.8 wt. % hydrogels for each of these crosslinked systems are shown FIG. 6.

The impact of the crosslinking reaction was evaluated by comparison of the tan δ (ratio of the shear loss modulus to the shear storage modulus) for two of the gels, the un-crosslinked and the DER 332 resin-crosslinked gels. An overlay of tan δ for the 4.8 wt. % gels are shown in FIG. 7. The flattening tan δ curve of the DER 332 resin-crosslinked hydrogel suggests that this gel is more rigid than the uncrosslinked hydrogel.

The hydrogels subjected to contact with buffer solution at body temperature were checked periodically for degradation via viscosity measurements and UV-Vis spectroscopy of the buffer layer. After more than 6 weeks, no signs of degradation were present in any of the hydrogels.

### EXAMPLE 6

Reduced, HMWK was prepared by placing 40 g of clean, dry human hair into a 1000 mL wide mouth glass reactor. 800 mL of a 0.8M solution of thioglycolic acid at pH 10.2 (adjusted with potassium hydroxide) was added and the mixture was stirred at room temperature under a nitrogen atmosphere for 18 hours. The solution was filtered and the liquid discarded. The hair was rinsed with copious amounts of deionized (DI) water, then placed back into the reactor. 400 mL of a 7M urea solution was added and the mixture was stirred at room temperature under a nitrogen atmosphere for 24 hours. After urea extraction, the solids were separated from the liquid by centrifugation. The liquid was added dropwise to a 10-fold volume excess of ethanol, thereby forming a keratin precipitate. The precipitated keratins were isolated by filtration and dried under vacuum. The resulting HMWK powder was ground by hand using a mortar and pestle.

### EXAMPLE 7

500 g of clean, dry human hair was placed in a 12000 mL round bottom flask. 8350 mL of 1 weight/volume percent of hydrogen peroxide was added and the reaction heated to reflux for 180 minutes. The hair was separated from the liquid by filtration and the liquid discarded. The hair was rinsed with copious amounts of water and allowed to air dry. 100 g of the dried, oxidized hair was placed in a 2000 mL round bottom flask. 1000 mL of 1M thioglycolic acid at pH 9 (adjusted with ammonium hydroxide) was added and the mixture was heated to 60° C. under a nitrogen atmosphere for 24 hours. After reductive extraction, the solids were separated from the liquid by centrifugation. The liquid was added dropwise to a 8-fold volume excess of ethanol, thereby forming a keratin precipitate. The precipitated HMWK keratins were isolated by filtration and dried under vacuum.

A solution was prepared by mixing 2 g of HMWK with 1 mL of 30% ammonium hydroxide and 10 mL of dimethyl sulfoxide. The solution was stirred and heated to ca. 75° C. to effect dissolution of the keratins. The solution was split into 4 volumes and placed into separate vials. To each of these 4 solutions was added 5, 10, 15, and 20 weight percent (relative to HMWK) of hexanediisocyanate, respectively, and 0.05, 0.1, 0.15, and 0.2 weight percent (relative to HMWK) of butyldilauryltin catalyst, respectively. The solutions were mixed using a vortex mixer, poured into separate petri dishes, and placed under a heat lamp. After 120 minutes of exposure, the samples were removed from the heat and peeled from the petri dishes. After curing, a small piece of each elastomer was immersed in a pH 7 aqueous buffer solution. After exposure to aqueous buffer for 48 hours, elastomers made with 5, 15, and 20 weight percent hexanediisocyanate were unchanged. The sample made with 10 weight percent hexanediisocyanate began to slowly hydrolyze after only 24 hours.

### EXAMPLE 8

4.5 g of the HMWK sample from Example 7 was dissolved in 2.25 mL of 30% ammonium hydroxide and 22.5 mL of dimethyl sulfoxide by heating to ca. 75° C. and stirred. The solution was split into 9 separate vials and used to prepare the solutions described in the following Table.

| | | Phthalic | | |
|---|---|---|---|---|
| | Terephthalic | Anhydride | DER ™ 332 | Sodium Acetate |
| Sample | Acid (grams) | (grams) | Resin (grams) | Catalyst (grams) |
| 1a | — | — | — | — |
| (control) | | | | |
| 2a | 0.025 | — | — | 0.005 |
| 2b | 0.050 | — | — | 0.005 |
| 3a | — | 0.025 | — | 0.005 |
| 3b | — | 0.050 | — | 0.005 |
| 4a | — | — | 0.025 | — |
| 4b | — | — | 0.050 | — |

The solutions were poured into separate petri dishes and placed under a heat lamp for ca. 240 minutes. After curing, a small piece of each elastomer was immersed in a pH 7 aqueous buffer solution. After exposure to aqueous buffer for 48 hours, elastomers 1a, 1a, 2b, 3a, and 3b had disintegrated and partially dissolved. After 6 days, elastomers 4a and 4b remained intact.

### EXAMPLE 9

175 grams of clean, dry human hair were extracted for 12 hours at room temperature with a solution of 265.9 grams of thioglycolic acid (TGA) diluted to 3.5 liters with deionized (DI) water. The pH of this solution was adjusted with 323 grams of potassium hydroxide (KOH) prior to use (pH 10.2). The extraction was performed with stirring under positive nitrogen pressure in a 4 liter glass reactor.

After extraction, the hair was separated by centrifugation followed by filtration and the liquid discarded. The reductively modified hair was rinsed with DI water to remove residual TGA and KOH then extracted with 3.2 liters of 7 molar aqueous urea solution. After 24 hours at room temperature, centrifugation and filtration isolated the extract. The aqueous protein solution was neutralized by addition of ca. 5 mL of hydrochloric acid and added to a 10-fold excess of ethanol. The resulting precipitate was rinsed with the following solutions:

1) 500 mL of 10/90 DI water/ethanol

2) 500 mL of ethanol

3) 500 mL of methanol

4) 500 mL of ethanol

The dehydrated keratin precipitate was place under vacuum to remove residual solvent, then ground into a fine powder.

4.0 grams of this keratin powder were mixed with a solution containing 0.4 grams of DER 736 resin (Aldrich Chemical Co., Milwaukee, Wis.), 0.5 grams of ethanol, and 75.1 grams of DI water. The ethanol was first used to dissolve the DER 736 resin, then added to the water. A thick suspension formed within 30 seconds.

This keratin suspension was poured into a 7×12×0.5 cm mold on a Teflon-lined glass plate. Over the hydrogel was placed a modified Telfa® pad (Beiersdorf Inc., Wilton, Conn.). The pad had been modified by removing the inner polymer liner, thereby exposing the inner cotton core. The pad was placed on the hydrogel with the cotton side down so that the gel would penetrate and bond to it. The outer polymer film of the pad would serve as a semipermeable barrier backing to the hydrogel dressing.

The mold was placed in an incubator at 37.5° C. for 24 hours. After curing, the dressing was peeled from the Teflon mold and remained intact. Digital photographs of the resulting dressing are shown below.

The hydrogel can be cured onto any adherent polymer or natural fiber backing. The intent of the backing is to provide structure so that the dressing can be easily handled, but also to serve as a barrier to moisture and pathogens. Many types of films are used for this purpose and the choice of backing can be made such that an optimal level of moisture retention and protection can be achieved. The level of hydration of the keratin hydrogel can also be varied to provide absorption of wound exudate. The keratin hydrogel is also capable of delivering biologically active compounds, inherent in keratins prepared from hair using the process described, thereby accelerating the wound healing and skin remodeling cascade. The keratin hydrogel can also be loaded with drugs, cells, and/or other proteins to be delivered to the wound bed.

### WOUND STUDIES

### EXAMPLE 10

175 grams of clean, dry human hair were extracted for 12 hours at room temperature with a solution of 265.9 grams of thioglycolic acid (TGA) diluted to 3.5 liters with deionized (DI) water. The pH of this solution was adjusted with 323 grams of potassium hydroxide (KOH) prior to use (pH 7.2). The extraction was performed with stirring under positive nitrogen pressure in a 4 liter glass reactor.

After extraction, the hair was separated by centrifugation followed by filtration and the liquid discarded. The reductively modified hair was rinsed with DI water to remove residual TGA and KOH then extracted with 3.2 liters of 7 molar aqueous urea solution. After 24 hours at room temperature, centrifugation and filtration isolated the extract. The aqueous protein solution was neutralized by addition of ca. 5 mL of hydrochloric acid and added to a 10-fold excess of ethanol. The resulting precipitate was rinsed with the following solutions:

5) 500 mL of 10/90 DI water/ethanol

6) 500 mL of ethanol

7) 500 mL of methanol

8) 500 mL of ethanol

The dehydrated keratin precipitate was place under vacuum to remove residual solvent, then ground into a fine powder. The keratin hydrogel was formed by adding 95.9 grams of ultrapure water to 4.01 grams of this powder (4 weight % solids) and mixing thoroughly.

### EXAMPLE 11

The keratin powder from Example 10 was utilized to make this hydrogel with one exception. The ultrapure water used to form the hydrogel contained 0.1 weight percent of low molecular weight keratins. In so doing, the hydrogel was “spiked” with matrix proteins that normally would not be present in large quantities.

### EXAMPLE 12

380 grams of clean, dry oxidized human hair were extracted for 24 hours at room temperature with a solution of 332.01 grams of TGA diluted to 3.46 liters with deionized (DI) water. The pH of this solution was adjusted to 9 with 400 mL of ammonium hydroxide (NH₄OH) prior to use). The extraction was performed with stirring under positive nitrogen pressure at 60° C. in a 4-liter glass reactor fitted with a cold water condenser.

After extraction, the hair was separated by centrifugation followed by filtration. The aqueous protein solution was added to a 10-fold excess of ethanol. The resulting precipitate was rinsed with excess ethanol and dried under vacuum, then ground into a fine powder.

A keratin-silicone elastomer was formed by dissolving 15 grams of this keratin powder into 45 grams of DI water. A photoinitiator was added (0.3 grams of anthraquinone-2-sulfonic acid sodium salt monohydrate) and a solution of 3 grams of vinyl-terminated silicone (catalogue no. DMS-V03; Gelest, Inc., Tullytown, Pa.) in 2 grams of isopropyl alcohol. After ca. 20 minutes of stirring, the thick solution was cast onto a Teflon coated glass plate and cured under a UV lamp for ca. 4 hours. The film was then dried under a heat lamp for ca. 1 hour. Samples of this film were cut into 1″×1″ squares to be used as wound dressings.

### EXAMPLE 13

380 grams of clean, dry oxidized human hair were extracted for 24 hours at room temperature with a solution of 332.01 grams of TGA diluted to 3.46 liters with deionized (DI) water. The pH of this solution was adjusted to 9 with 400 mL of ammonium hydroxide (NH₄OH) prior to use). The extraction was performed with stirring under positive nitrogen pressure at 60° C. in a 4-liter glass reactor fitted with a cold water condenser.

After extraction, the hair was separated by centrifugation followed by filtration. The aqueous protein solution was added to a 10-fold excess of ethanol. The resulting precipitate was rinsed with excess ethanol and dried under vacuum, then ground into a fine powder.

20 grams of this keratin powder was dissolved in 60 grams of DI water, then added 0.2 grams of DER 736 resin (Aldrich, Milwaukee, Wis.) in 10 grams of isopropyl alcohol. The pH of the solution was adjusted to 7.0 by addition of 3 drops of NH₄OH. The solutions was stirred at room temperature for ca. 45 minutes, then poured into a mold on Teflon coated glass. The film was cured using slight heating for ca. 7 hours. Samples of this film were cut into 1″×1″ squares to be used as wound dressings.

### EXAMPLE 14

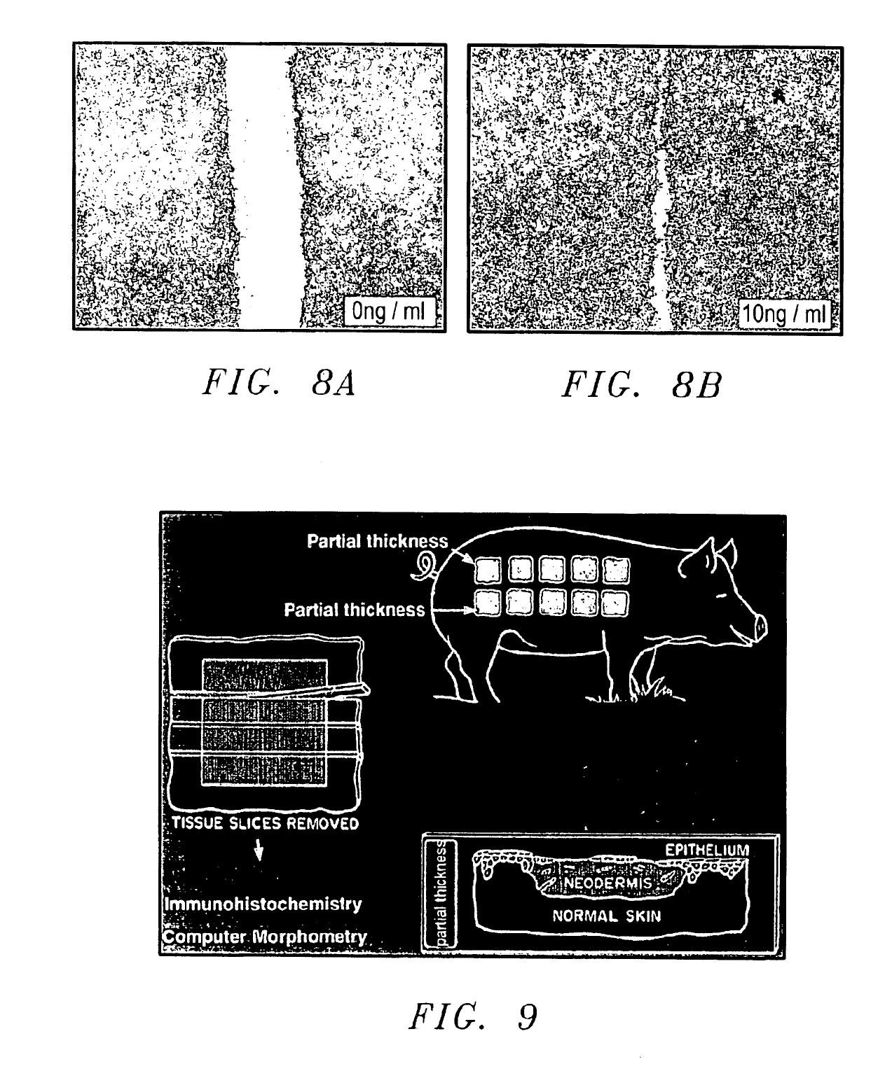

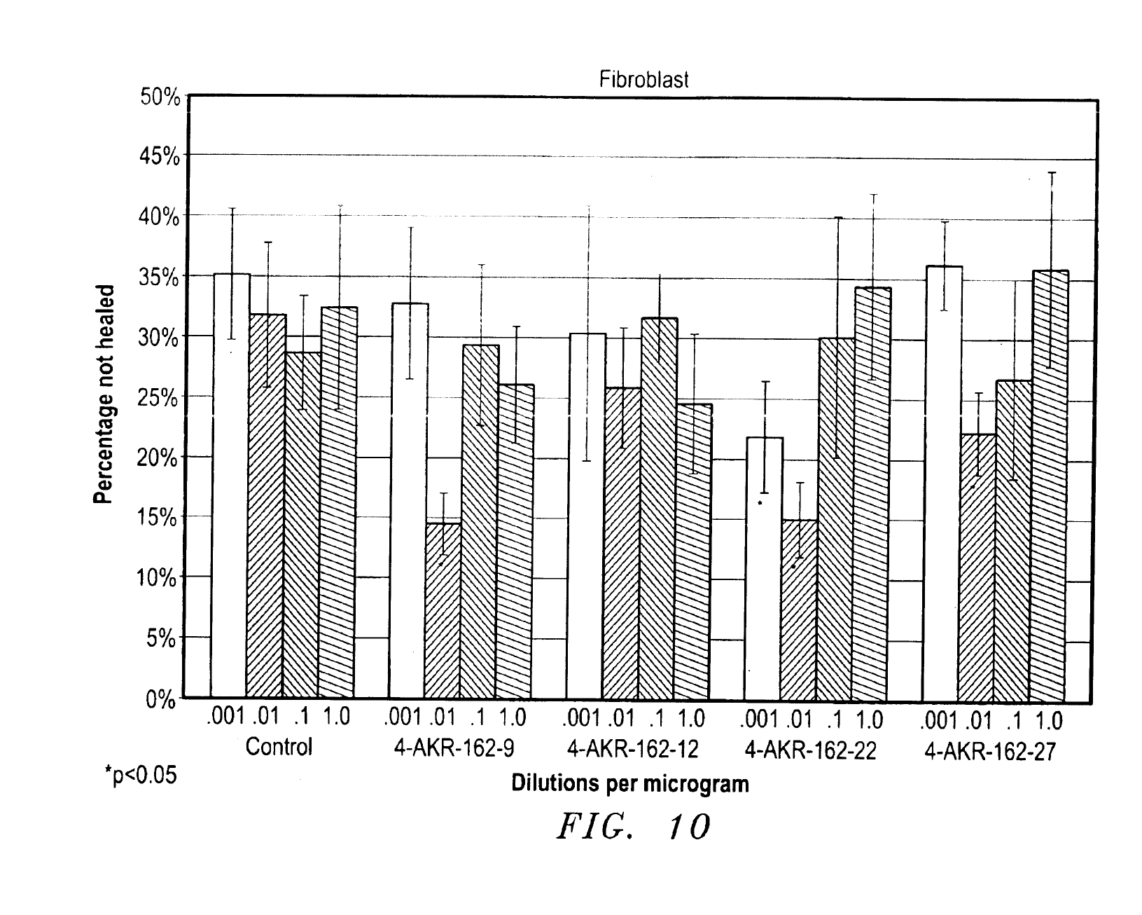

Physiologic stimulation with human hair extracts and extracts from keratin biomaterials were used to establish evidence of biological responses. Second or third passage cells were grown in serum containing media to ˜80% confluence on fibronectin-coated Nunc single chamber slides. Cells were serum starved for 6 hours and then 6 wounds (scratch assays; see FIG. 8) were created per slide. Defined media (minus serum) with and without keratins were introduced, and cells were returned to the incubator to heal for 18 to 48 hours followed by fixation in 4% paraformaldehyde and staining with hematoxylin. During this healing interval, cells attempt to close the defect with mitosis and migration.

An initial dose response series was employed to screen for effective and deleterious concentrations of keratins. The size of the wounded area was assessed using Image Proplus software (Media Cybernetics) and comparisons were made to determine the amount of healing.

Fibroblast cultures showed biological activation by some of the keratin solutions. Increases in healing as high as 230% over the control were measured. Concentrations of keratin as low as 0.001 mg/mL showed statistically significant (p<0.05) increases in healing.

A-431 cell cultures (a mouse epithelial cell line) showed biological activation by most of the keratin solutions. Increases in healing as high as 230% over the control were measured. Concentrations of keratin as low as 0.001 mg/mL showed statistically significant (p<0.05) increases in healing.

### EXAMPLE 15

A Yorkshire pig (Sus scrofa) weighing approximately 75-100 lbs. was used. Partial-thickness excisions (open wound beds) were created with a Padgett dermatome calibrated to shave a thickness of ca. 1200 microns. FIG. 1 illustrates wound placement patterns and depicts techniques used in this study to examine the histological specimens at different time points during wound repair.

The initial pig received a series of partial thickness excisions created with a Padgett Dermatome. The wounds that were destined to become the 7-day wounds (10 of these) were created on the initial day of the experiment. The wounds that were destined to become the 4-day wounds were created 3 days after the initiation of the study (10 of these). When the pig was euthanized at the conclusion of the experiment, all the wounds were removed for analysis. Formulations were applied to each wound on a daily basis. Wounds were then wrapped in semi-occlusive bandage (Op-site) to ensure that the formulation remained in position and to prevent mixing of the various topical formulations. The semi-occlusive bandage also acted to facilitate healing by maintaining a moist environment. Wound treatments were as follows: control (only occlusive dressing), Gel 4-AKR-178-2 (Example 10), Gel 4-AKR-178-12 (Example 11), Film 1-SEB-113-2 (Example 13), Film 7-AR-44-2 (Example 12). In this initial study all wound treatments were performed in duplicate. Thus the number of wounds were two per day per treatment group.

Quantitative outcomes included epidermal resurfacing, the influx (cell density) of neutrophils or monocyte/macrophages, and capillary density. Morphological differences between the various dosages and compounds, if present, were detected by a number of routine and special stains performed on histological specimens. The wounds were stained with Gomori's trichrome which is useful for delineating the wound margins. The proliferating cells (keratinocytes or fibroblasts) were identified using specific immunomarkers (PCNA—proliferating cell nuclear antigen). The density of neutrophils and monocytes and macrophages were quantified manually using standard morphological indicators such as nuclear morphology and size differentials.

The status of the vascular network within wounds is important for several reasons. During the acute wound healing period, endothelial cells can potentially regulate the inflammatory response by controlling the transmigration of neutrophils and monocytes into the wound bed. In addition, angiogenesis is a reliable indicator of the healing kinetics within a wound bed. If wounds mature normally, capillary density diminishes. If warranted, immunohistological evaluations were performed retrospectively when tissues were removed. Capillary density was assessed using selective immunostaining of Factor VIII+endothelial cells followed by computerized quantitative morphometric analysis. Wounds were collected to evaluate for short-term effects such as proliferation, migration of epidermal cells, fibroblasts and endothelial cells.

Histological Impressions

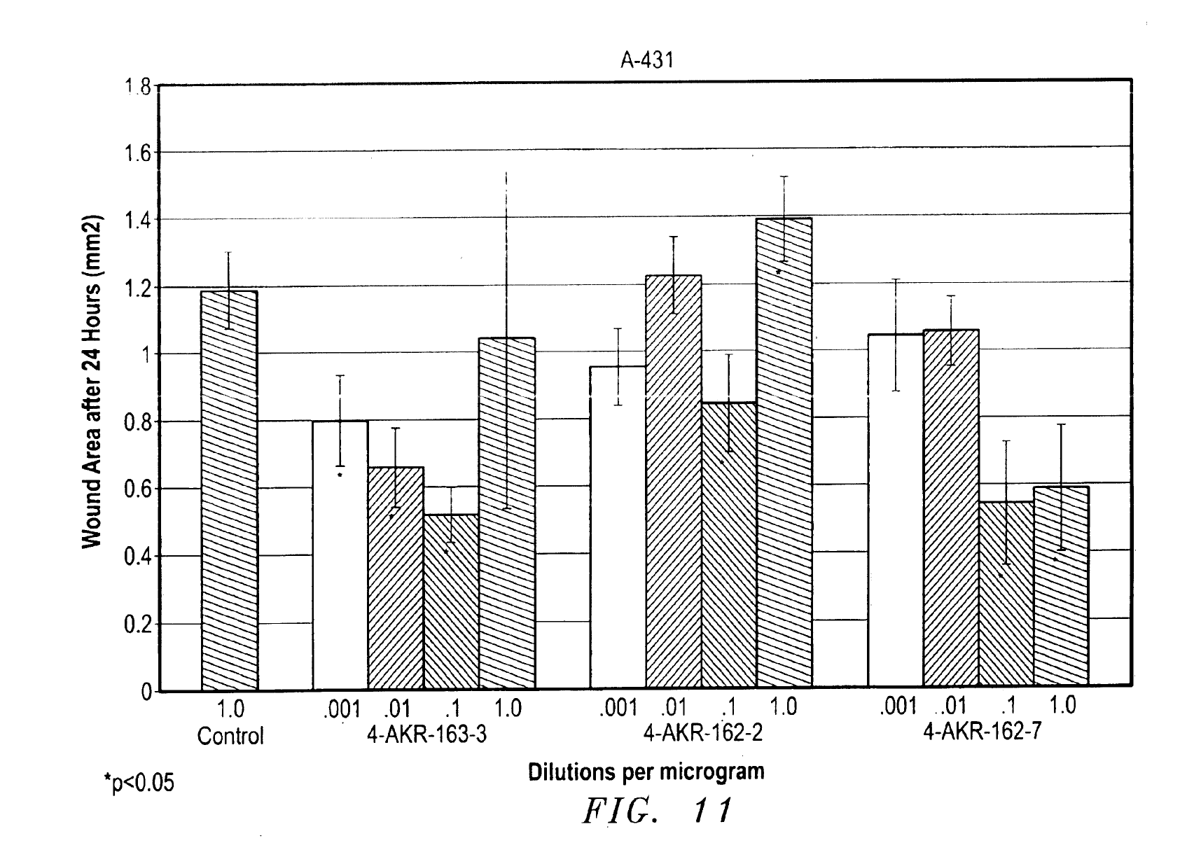

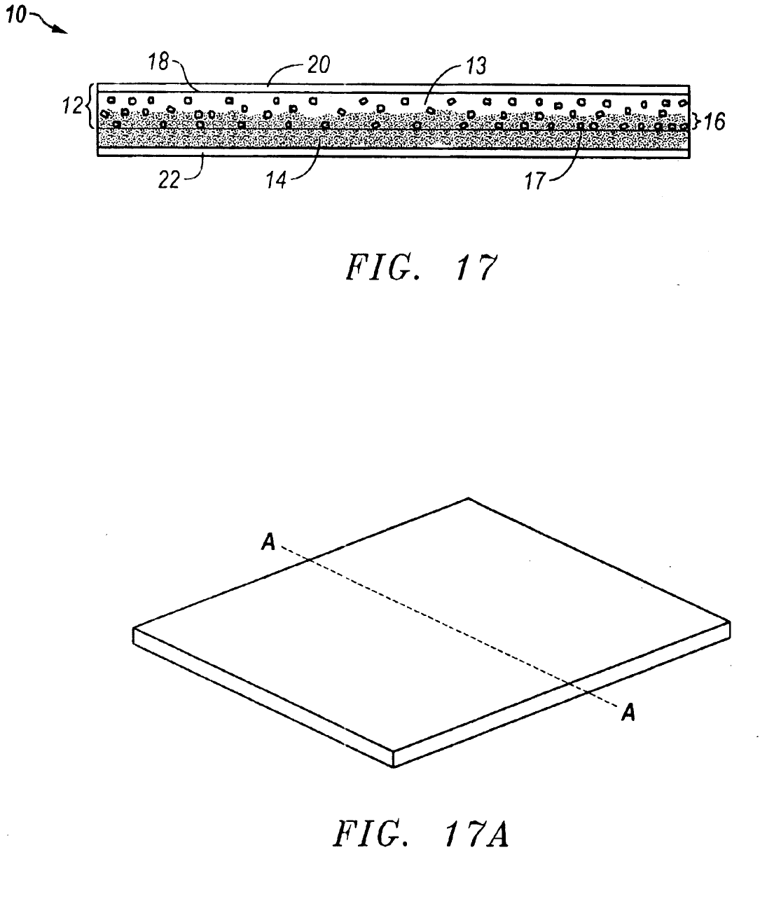

Referring to FIGS. 12-16, the most notable feature histologically was the hypertrophic response of the epidermis for both of the gels and for both of the films. This finding was consistently observed in all of the sections except for the controls. It definitely appeared that there were bioactive ingredients in each of the 4 formulations, causing a gross biological outcome change. This feature was most prominent in the 7-day wounds. While all porcine wounds were 100% resurfaced at this timepoint, the treatments exhibited their greatest impact on the morphological appearance of the dermal-epidermal junction. In control wounds the dermal-epidermal junction was largely flat and unremarkable as it is in typical scars. By contrast, in the test wounds, there were numerous downward and upward projections of the epidermal rete ridges and the numbers of epidermal layers appeared mostly increased. This suggests that the newly healed epidermis in the test wounds would be more likely to adhere to the surface of the dermis and would be less likely to shear off and be as fragile as normal. These results indicate that keratinocytes of the epidermis and epidermal appendages are especially responsive to certain cytokines and mediators in these formulations.

Quantitative Morphometric Analysis:

Re-Epithelialization: At 4 days, several of the wounds appeared remarkably more resurfaced than the control. The variance between the duplicate samples was widely divergent, probably due to some variations in the original thickness of the wounds. However, when taken with the histological evidence, we can say with confidence that the formulations have had an affect on the epidermis. At 7 days essentially all the wounds are 100% resurfaced as would be expected.

Dermal Depth: At day 4, the control samples were not measurable so we had no true basis for comparison. From the day 7 material, the two duplicate control samples were very close in depth and suggest a reasonable baseline for comparison.

The following table summarizes the epithelial resurfacing data:

| Treatment Group | Average % Healed |
|---|---|
| Hydrogel A | 39.80 |
| Hydrogel B | 16.45 |
| Elastomer A | 27.15 |
| Elastomer B | 25.40 |
| Control | 11.80 |

### EXAMPLE 16

Procedure. The two pigs used in this study received a series of partial thickness excisions and full-thickness excisions created with a Padgett Dermatome. The wounds that were destined to become the 14-day wounds were created on the initial day of the experiment. The wounds that were destined to become the 7-day wounds were created 7 days after the initiation of the study. When the pig was euthanized at the conclusion of the experiment, all the wounds were removed for analysis. The right side of the pig was given the partial thickness wounds and the left side of the pig received the full thickness wounds. The pigs were given preanesthetics of atropine, ketamine and acepromazine. They were maintained on inhalation anesthesia of Nitrous oxide and oxygen. Postoperative medications included Buprenex every 12 hours for analgesia and cephelexin to prevent infection. Keratin formulations were applied to each wound on a daily basis for a period of 5 days of continuous treatment. Wounds were then wrapped in semi-occlusive bandage (Op-site) to ensure that the formulation remained in position and to prevent mixing among topical formulations and to prevent mechanical trauma to the healing wounds. The semi-occlusive bandage also acted to facilitate healing by maintaining a moist environment. Wound treatments were either the gel formulation or no treatment. In this initial study all wound treatments were performed in duplicate. Thus the numbers of wounds were two per day per treatment group per pig. N=4 wounds in this experiment for each treatment.

Porcine Observations: The two pigs appeared to tolerate the formulation and the wounding quite well. There were no obvious signs of infection observed either grossly or was their evidence of infection observed in the histological sections. The semi-occlusive dressing remained firmly in place and no wounds were subjected to unexpected desiccation.

Ouantitative Morphometric Analysis: The quantitative morphometric analyses were performed using an Olympus AH2 light microscope that was interfaced to a Pixcera digital camera. Images in jpg format were collected and analyzed using Image Proplus software (Media Cybernetics). Analysis of resurfacing was performed on histological sections that were stained with Gomori's one-step Trichrome. Three random sections from each wound were positioned on glass slides. The total distance on one end of the wound to the other end is assessed in microns, and then the distance of new epithelial coverage was added. This represents the sum of the epithelial lengths growing out from the wounds edges as well as the outgrowths from islands of new epithelium that emanate from epidermal remnants such as hair follicles and sweat ducts.

Re-Epithelialization: Some notable differences were observed among the 8 different treatment groups. In the partial thickness wounds after 7 days of healing, the wounds were better resurfaced in the control group as opposed to the gel treated group. However, when the experiment was allowed to proceed further and the tissue was harvested after 14 days, it was apparent that the mean resurfacing was 91% in the gel treatment and only 56% resurfaced in the control gel group. These data are summarized in the graph in FIG. 1.

Neovascularization: Data from the 7-day treatment groups was fairly unremarkable. While the keratin gel in the partial treatment group appeared to show a trend toward a statistically significant difference between keratin treatment and the control wounds, it would require additional pigs to confirm whether this was indeed true or was merely the result of chance. In the wounds that were treated for 14 days, the keratin gel treatment appeared to stimulate a robust angiogenic response in the partial thickness group of wounds. This difference did not reach statistical significance in the two pigs that were studied (N=4 wounds). These data are summarized in the graph in FIG. 18. The keratin gel treatment was unremarkable in the full-thickness wounds due to the immaturity of these wounds.

Persons of ordinary skill in the art will recognize that many modifications may be made to the present invention without departing from the spirit and scope of the present invention. The embodiment described herein is meant to be illustrative only and should not be taken as limiting the invention, which is defined in the claims.

I claim:

1. A tissue defect dressing comprising a proteinaceous salve comprising keratinaceous molecules networked by interkeratin associations other than glutaraldehyde, the interkeratin associations comprising covalent bonds between the keratinaceous molecules comprising primarily other than disulfide bonds, said interkeratin associations producing a keratinaceous network.

2. The tissue defect dressing of claim 1 wherein said keratinaceous molecules consist essentially of water soluble keratinaceous molecules.

3. The tissue defect dressing of claim 1 further comprising one or more added bioactive component.

4. The tissue defect dressing of claim 2 further comprising one or more added bioactive component.

5. The tissue defect dressing of claim 1 wherein the keratinaceous molecules are α-keratinaceous molecules.

6. The tissue defect dressing of claim 2 wherein the keratinaceous molecules are α-keratinaceous molecules.

7. The tissue defect dressing of claim 4 wherein the keratinaceous molecules are α-keratinaceous molecules.

8. The tissue defect dressing of claim 1 comprising a cohesive tissue defect dressing.

9. The tissue defect dressing of claim 6 comprising a cohesive tissue defect dressing.

10. The tissue defect dressing of claim 1 further comprising a support adapted to support the proteinaceous salve.

11. The tissue defect dressing of claim 2 further comprising a support adapted to support the proteinaceous salve.

12. The tissue defect dressing of claim 6 further comprising a support adapted to support the proteinaceous salve.

13. The tissue defect dressing of claim 7 further comprising a support adapted to support the proteinaceous salve.

14. The tissue defect dressing of claim 8 further comprising a support adapted to support the proteinaceous salve and render the tissue defect dressing cohesive.

15. The tissue defect dressing of claim 9 further comprising a support adapted to support the proteinaceous salve and render the tissue defect dressing cohesive.

16. The tissue defect dressing of claim 10 wherein the support comprises a sheet of permeable material having an inner surface abutting the proteinaceous salve and an outer surface abutting a barrier material.

17. The tissue defect dressing of claim 11 wherein the support comprises a sheet of permeable material having an inner surface abutting the proteinaceous salve and an outer surface abutting a barrier material.

18. The tissue defect dressing of claim 12 wherein the support comprises a sheet of permeable material having an inner surface abutting the proteinaceous salve and an outer surface abutting a barrier material.

19. The tissue defect dressing of claim 13 wherein the support comprises a sheet of permeable material having an inner surface abutting the proteinaceous salve and an outer surface abutting a barrier material.

20. The tissue defect dressing of claim 14 wherein the support comprises a sheet of permeable material having an inner surface abutting the proteinaceous salve and an outer surface abutting a barrier material.

21. The tissue defect dressing of claim 15 wherein the support comprises a sheet of permeable material having an inner surface abutting the proteinaceous salve and an outer surface abutting a barrier material.

22. The tissue defect dressing of claim 1 wherein the proteinaceous salve comprises a hydrogel comprising water retained in the proteinaceous network.

23. The tissue defect dressing of claim 2 wherein the proteinaceous salve comprises a hydrogel comprising water retained in the proteinaceous network.

24. The tissue defect dressing of claim 6 wherein the proteinaceous salve comprises a hydrogel comprising water retained in the proteinaceous network.

25. A tissue defect dressing comprising a support adapted to support a proteinaceous salve, the proteinaceous salve comprising water soluble α-keratinaceous molecules networked by interkeratin associations other than glutaraldehyde, the interkeratin associations comprising covalent bonds between the water soluble α-keratinaceous molecules comprising primarily other than disulfide bonds, said interkeratin associations producing a keratinaceous network.

26. The tissue defect dressing of claim 18 wherein the proteinaceous salve comprises a hydrogel comprising water retained in the proteinaceous network.

27. The tissue defect dressing of claim 2 wherein the keratinaceous material is derived from human hair.

28. The tissue defect dressing of claim 22 wherein the keratinaceous material is derived from human hair.

29. The tissue defect dressing of claim 23 wherein the keratinaceous material is derived from human hair.

30. The tissue defect dressing of claim 24 wherein the keratinaceous material is derived from human hair.

31. The tissue defect dressing of claim 25 wherein the proteinaceous material is derived from human hair.

32. The tissue defect dressing of claim 26 wherein the proteinaceous material is derived from human hair.

33. The tissue defect dressing of claim 1 wherein the covalent bonds comprise primarily interprotein crosslinks comprising first covalent bonds between first functional groups on a plurality of molecules of a crosslinking agent and first reactive pendant groups on a plurality of first keratin molecules and second covalent bonds between second functional groups on a plurality of molecules of the crosslinking agent and second reactive pendant groups on a plurality of second keratin molecules.

34. The tissue defect dressing of claim 2 wherein the covalent bonds comprise primarily interprotein crosslinks comprising first covalent bonds between first functional groups on a plurality of molecules of a crosslinking agent and first reactive pendant groups on a plurality of first water soluble keratin molecules and second covalent bonds between second functional groups on a plurality of molecules of the crosslinking agent and second reactive pendant groups on a plurality of second water soluble keratin molecules.

35. The tissue defect dressing of claim 6 wherein the covalent bonds comprise primarily interprotein crosslinks comprising first covalent bonds between first functional groups on a plurality of molecules of a crosslinking agent and first reactive pendant groups on a plurality of first water soluble keratin molecules and second covalent bonds between second functional groups on a plurality of molecules of the crosslinking agent and second reactive pendant groups on a plurality of second water soluble keratin molecules.

36. The tissue defect dressing of claim 12 wherein the covalent bonds comprise primarily interprotein crosslinks comprising first covalent bonds between first functional groups on a plurality of molecules of a crosslinking agent and first reactive pendant groups on a plurality of first water soluble keratin molecules and second covalent bonds between second functional groups on a plurality of molecules of the crosslinking agent and second reactive pendant groups on a plurality of second water soluble keratin molecules.

37. The tissue defect dressing of claim 18 wherein the covalent bonds comprise primarily interprotein crosslinks comprising first covalent bonds between first functional groups on a plurality of molecules of a crosslinking agent and first reactive pendant groups on a plurality of first water soluble keratin molecules and second covalent bonds between second functional groups on a plurality of molecules of the crosslinking agent and second reactive pendant groups on a plurality of second water soluble keratin molecules.

38. The tissue defect dressing of claim 22 wherein the covalent bonds comprise primarily interprotein crosslinks comprising first covalent bonds between first functional groups on a plurality of molecules of a crosslinking agent and first reactive pendant groups on a plurality of first water soluble keratin molecules and second covalent bonds between second functional groups on a plurality of molecules of the crosslinking agent and second reactive pendant groups on a plurality of second water soluble keratin molecules.

39. The tissue defect dressing of claim 23 wherein the covalent bonds comprise primarily interprotein crosslinks comprising first covalent bonds between first functional groups on a plurality of molecules of a crosslinking agent and first reactive pendant groups on a plurality of first water soluble keratin molecules and second covalent bonds between second functional groups on a plurality of molecules of the crosslinking agent and second reactive pendant groups on a plurality of second water soluble keratin molecules.

40. The tissue defect dressing of claim 24 wherein the covalent bonds comprise primarily interprotein crosslinks comprising first covalent bonds between first functional groups on a plurality of molecules of a crosslinking agent and first reactive pendant groups on a plurality of first water soluble keratin molecules and second covalent bonds between second functional groups on a plurality of molecules of the crosslinking agent and second reactive pendant groups on a plurality of second water soluble keratin molecules.

41. A tissue defect dressing comprising:
a support adapted to support a proteinaceous salve comprising water soluble α-keratinaceous molecules networked by interkeratin associations other than glutaraldehyde, the interkeratin associations comprising covalent bonds between the water soluble α-keratinaceous molecules comprising primarily other than disulfide bonds, said interkeratin associations producing a keratinaceous network;
wherein the covalent bonds comprise primarily interprotein crosslinks comprising first covalent bonds between first functional groups on a plurality of molecules of a crosslinking agent and first reactive pendant groups on a plurality of first water soluble keratin molecules and second covalent bonds between second functional groups on a plurality of molecules of the crosslinking agent and second reactive pendant groups on a plurality of second water soluble keratin molecules.

42. The tissue defect dressing of claim 26 wherein the covalent bonds consist essentially of interprotein crosslinks comprising first covalent bonds between first functional groups on a plurality of molecules of a crosslinking agent and first reactive pendant groups on a plurality of first water soluble keratins and second covalent bonds between second functional groups on a plurality of molecules of said crosslinking agent and second reactive pendant groups on a plurality of second water soluble keratins.

43. The tissue defect dressing of claim 27 wherein the covalent bonds consist essentially of interprotein crosslinks comprising first covalent bonds between first functional groups on a plurality of molecules of a crosslinking agent and first reactive pendant groups on a plurality of first water soluble keratins and second covalent bonds between second functional groups on a plurality of molecules of said crosslinking agent and second reactive pendant groups on a plurality of second water soluble keratins.

44. The tissue defect dressing of claim 32 wherein the covalent bonds consist essentially of interprotein crosslinks comprising first covalent bonds between first functional groups on a plurality of molecules of a crosslinking agent and first reactive pendant groups on a plurality of first water soluble keratins and second covalent bonds between second functional groups on a plurality of molecules of said crosslinking agent and second reactive pendant groups on a plurality of second water soluble keratins.

45. The tissue defect dressing of claim 34 wherein the first and second functional groups comprise groups selected from the group consisting of alkoxide groups, allyl groups, vinyl groups, hydroxyl groups, amine groups, aldehyde groups, isocyanate groups, ester groups, and anhydride groups.

46. The tissue defect dressing of claim 38 wherein the first and second functional groups comprise groups selected from the group consisting of alkoxide groups, allyl groups, vinyl groups, hydroxyl groups, amine groups, aldehyde groups, isocyanate groups, ester groups, and anhydride groups.

47. The tissue defect dressing of claim 39 wherein the first and second functional groups comprise groups selected from the group consisting of alkoxide groups, allyl groups, vinyl groups, hydroxyl groups, amine groups, aldehyde groups, isocyanate groups, ester groups, and anhydride groups.

48. The tissue defect dressing of claim 40 wherein the first and second functional groups comprise groups selected from the group consisting of alkoxide groups, allyl groups, vinyl groups, hydroxyl groups, amine groups, aldehyde groups, isocyanate groups, ester groups, and anhydride groups.

49. The tissue defect dressing of claim 41 wherein the first and second functional groups comprise groups selected from the group consisting of alkoxide groups, allyl groups, vinyl groups, hydroxyl groups, amine groups, aldehyde groups, isocyanate groups, ester groups, and anhydride groups.

50. The tissue defect dressing of claim 42 wherein the first and second functional groups comprise groups selected from the group consisting of alkoxide groups, allyl groups, vinyl groups, hydroxyl groups, amine groups, aldehyde groups, isocyanate groups, ester groups, and anhydride groups.

51. The tissue defect dressing of claim 43 wherein the first and second functional groups comprise groups selected from the group consisting of alkoxide groups, allyl groups, vinyl groups, hydroxyl groups, amine groups, aldehyde groups, isocyanate groups, ester groups, and anhydride groups.

52. The tissue defect dressing of claim 44 wherein the first and second functional groups comprise groups selected from the group consisting of alkoxide groups, allyl groups, vinyl groups, hydroxyl groups, amine groups, aldehyde groups, isocyanate groups, ester groups, and anhydride groups.

53. The tissue defect dressing of claim 38 wherein the reactive pendant groups comprise groups selected from the group consisting of hydroxyl groups, thiol groups, reactive amine groups, and epoxides.

54. The tissue defect dressing of claim 41 wherein the reactive pendant groups comprise groups selected from the group consisting of hydroxyl groups, thiol groups, reactive amine groups, and epoxides.

55. The tissue defect dressing of claims 42 wherein the reactive pendant groups comprise groups selected from the group consisting of hydroxyl groups, thiol groups, reactive amine groups, and epoxides.

56. The tissue defect dressing of claims 43 wherein the reactive pendant groups comprise groups selected from the group consisting of hydroxyl groups, thiol groups, reactive amine groups, and epoxides.

57. The tissue defect dressing of claims 44 wherein the reactive pendant groups comprise groups selected from the group consisting of hydroxyl groups, thiol groups, reactive amine groups, and epoxides.

58. The tissue defect dressing of claim 51 wherein the reactive pendant groups comprise groups selected from the group consisting of hydroxyl groups, thiol groups, reactive amine groups, and epoxides.

59. The tissue defect dressing of claim 52 wherein the reactive pendant groups comprise groups selected from the group consisting of hydroxyl groups, thiol groups, reactive amine groups, and epoxides.

60. The tissue defect dressing of claim 38 wherein at least one of a moiety selected from the group consisting of the first functional groups, the first reactive pendant groups, the second functional groups, and the second reactive pendant groups comprises an epoxide.

61. The tissue defect dressing of claim 41 wherein at least one of a moiety selected from the group consisting of the first functional groups, the first reactive pendant groups, the second functional groups, and the second reactive pendant groups comprises an epoxide.

62. The tissue defect dressing of claim 42 wherein at least one of a moiety selected from the group consisting of the first functional groups, the first reactive pendant groups, the second functional groups, and the second reactive pendant groups comprises an epoxide.

63. The tissue defect dressing of claim 43 wherein at least one of a moiety selected from the group consisting of the first functional groups, the first reactive pendant groups, the second functional groups, and the second reactive pendant groups comprises an epoxide.

64. The tissue defect dressing of claim 44 wherein at least one of a moiety selected from the group consisting of the first functional groups, the first reactive pendant groups, the second functional groups, and the second reactive pendant groups comprises an epoxide.

65. The tissue defect dressing of claim 51 wherein at least one of a moiety selected from the group consisting of the first functional groups, the first reactive pendant groups, the second functional groups, and the second reactive pendant groups comprises an epoxide.

66. The tissue defect dressing of claim 52 wherein at least one of a moiety selected from the group consisting of the first functional groups, the first reactive pendant groups, the second functional groups, and the second reactive pendant groups comprises an epoxide.

67. The tissue defect dressing of claim 34 wherein the crosslinking agent is a heterogeneous crosslinking agent.

68. The tissue defect dressing of claim 35 wherein the crosslinking agent is a heterogeneous crosslinking agent.

69. The tissue defect dressing of claim 36 wherein the crosslinking agent is a heterogeneous crosslinking agent.

70. The tissue defect dressing of claim 39 wherein the crosslinking agent is a heterogeneous crosslinking agent.

71. The tissue defect dressing of claim 41 wherein the crosslinking agent is a heterogeneous crosslinking agent.

72. The tissue defect dressing of claim 49 wherein the crosslinking agent is a heterogeneous crosslinking agent.

73. The tissue defect dressing of claim 71 wherein the heterogeneous crosslinking agent produces crosslinks comprising the following structure: 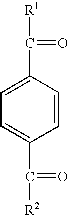
wherein R¹ and R² independently are amino acid residues of separate proteinaceous molecules, said amino acid residues being selected from the group consisting of cysteine, arginine, serine, lysine, asparagine, glutamine, tyrosine, tryptophan, and histidine.

74. The tissue defect dressing of claim 71 wherein the heterogeneous crosslinking agent produces crosslinks comprising the following structure: 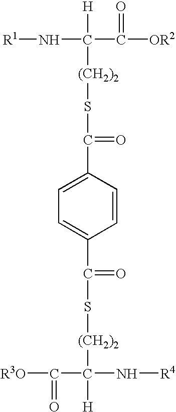
wherein R¹ and R² independently are amino acid residues of separate proteinaceous molecules, said amino acid residues being selected from the group consisting of cysteine, arginine, serine, lysine, asparagine, glutamine, tyrosine, tryptophan, and histidine.

75. The tissue defect dressing of claim 71 wherein the heterogeneous crosslinking agent produces crosslinks comprising the following structure: 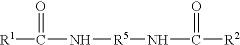
wherein
R¹ and R² independently are amino acid residues of separate water soluble keratins, said amino acid residues being selected from the group consisting of glutamic acid and aspartic acid; and,
R⁵ is selected from the group consisting of alkoxy groups, alkylene groups, and alkenyl groups having from about 1 to about 50 carbon atoms, alone, or in combination with cyclic alkyl groups or aromatic groups.

76. The tissue defect dressing of claim 71 wherein the heterogeneous crosslinking agent produces crosslinks comprising the following structure: 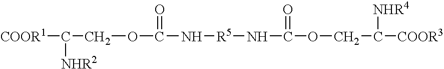
wherein
R¹ and R² are the remainder of a first water soluble keratin;
R³ and R⁴ are the remainder of a second water soluble keratin; and,
R⁵ is selected from the group consisting of alkoxy groups, alkylene groups, and alkenyl groups having from about 1 to about 50 carbon atoms, alone, or in combination with cyclic alkyl groups or aromatic groups.

77. The tissue defect dressing of claim 71 wherein the heterogeneous crosslinking agent produces crosslinks comprising the following structure: 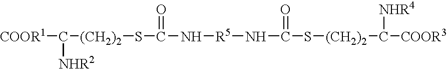
wherein
R¹ and R² are the remainder of a first water soluble keratin; and,
R³ and R⁴ are the remainder of a second water soluble keratin; and,
R⁵ is selected from the group consisting of alkoxy groups, alkylene groups, and alkenyl groups having from about 1 to about 50 carbon atoms, alone, or in combination with cyclic alkyl groups or aromatic groups.

78. The tissue defect dressing of claim 71 wherein the heterogeneous crosslinking agent produces crosslinks comprising the following structure: 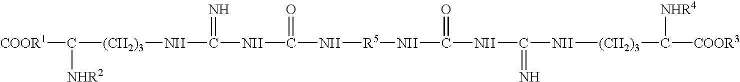
wherein
R¹ and R² are the remainder of a first water soluble keratin;
R³ and R⁴ are the remainder of a second water soluble keratin; and,
R⁵ is selected from the group consisting of alkoxy groups, alkylene groups, and alkenyl groups having from about 1 to about 50 carbon atoms, alone, or in combination with cyclic alkyl groups or aromatic groups.

79. The tissue defect dressing of claim 71 wherein the heterogeneous crosslinking agent produces crosslinks comprising the following structure: 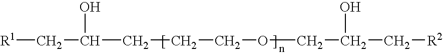
wherein
n is from about 1 to about 50; and,
R¹ and R² independently are amino acid residues of separate water soluble keratins, the residues being selected from the group consisting of cysteine, arginine, serine, lysine, asparagine, glutamine, tyrosine, tryptophan, and histidine.

80. The tissue defect dressing of claim 71 wherein the heterogeneous crosslinking agent produces crosslinks comprising the following structure: 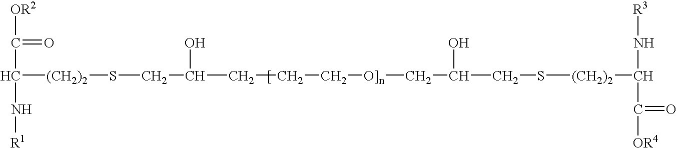
wherein
n is from about 1 to about 50;
R¹ and R² are the remainder of a first water soluble keratin; and,
R³ and R⁴ are the remainder of a second water soluble keratin.

81. The tissue defect dressing of claim 71 wherein the heterogeneous crosslinking agent produces crosslinks comprising the following structure: 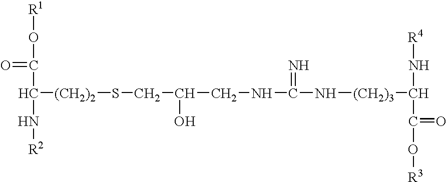
wherein
R¹ and R² are the remainder of a first water soluble keratin; and
R³ and R⁴ are the remainder of a second water soluble keratin.

82. The tissue defect dressing of claim 71 wherein the heterogeneous crosslinking agent produces crosslinks comprising the following structure: 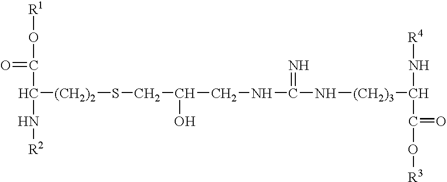
wherein
R¹ and R² are the remainder of a first water soluble keratin; and
R³ and R⁴ are the remainder of a second water soluble keratin.

83. The tissue defect dressing of claim 71 wherein the heterogeneous crosslinking agent comprises silicone.

84. The tissue defect dressing of claim 83 wherein the heterogeneous crosslinking agent has the following general structure: 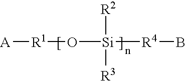
wherein
n is from about 1 to about 50; and,
A and B are the remainder of first and second protein molecules;
at least two of R¹, R², R³, and R⁴ comprise at least one reactive functionality comprising at least one reactive moiety selected from the group consisting of a reactive unsaturated carbon-carbon bond, a reactive oxygen containing group, a reactive nitrogen containing group, and a reactive sulfur-containing group.

85. The tissue defect dressing of claim 84 wherein
R¹, R², R³, and R⁴ are selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

86. The tissue defect dressing of claim 84 wherein
R¹ and R⁴ independently are selected from the group consisting of hydrogen, linear, branched or cyclic alkyl groups having from about 1 to about 6 carbon atoms, alkenyl groups having from about 2 to about 6 carbon atoms, hydrido groups, alkoxy groups comprising one or more alkyl groups having a total of from about 1 to about 6 carbon atoms, hydroxy groups, alkylamine groups, alkylmercapto groups, acrylate groups, methacrylate groups, halo groups, acetoxy groups, and epoxy groups;
R² and R³ independently are selected from the group consisting of hydrogen, cycloalkyl groups, vinyl groups, hydrido groups, trifluoroalkyl groups, phenyl groups, alkyl groups, alkoxy groups, alkylmercapto groups, and alkylamine groups; provided that, when one of R² or R³ is a vinyl group, the other of R or R is a group other than a hydrido group; and, when one of R² or R³ is a hydrido group, the other of R² or R³ is a group other than a vinyl group.

87. The tissue defect dressing of claim 84 wherein at least one of R² and R³ is an alkyl group.

88. The tissue defect dressing of claim 84 wherein at least one of R² and R³ is a methyl group.

89. The tissue defect dressing of claim 85 wherein at least one of R² and R³ is an alkyl group.

90. The tissue defect dressing of claim 85 wherein at least one of R² and R³ is a methyl group.

91. The tissue defect dressing of claim 86 wherein at least one of R² and R³ is an alkyl group.

92. The tissue defect dressing of claim 86 wherein at least one of R² and R³ is a methyl group.

93. The tissue defect dressing of claim 71 wherein the heterogeneous crosslinking agent produces crosslinks comprising the following structure: 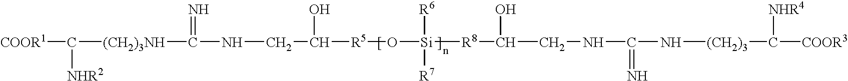
wherein
n is from about 1 to about 50;
R¹ and R² are a remainder of a first keratin molecule;
R³ and R⁴ is a remainder of a second keratin molecule; and,
R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

94. The tissue defect dressing of claim 93 wherein at least one of R⁶ and R⁷ is a methyl group.

95. The tissue defect dressing of claim 93 wherein R⁵ and R⁸ are selected from the group consisting of alkyl groups having from about 1 to about 6 carbon atoms and dimethylsiloxy groups.

96. The tissue defect dressing of claim 94 wherein R⁵ and R⁸ are selected from the group consisting of alkyl groups having from about 1 to about 6 carbon atoms and dimethylsiloxy groups.

97. The tissue defect dressing of claim 93 wherein R⁵ and R⁸ comprise n-propoxypropyl groups.

98. The tissue defect dressing of claims 94 wherein R⁵ and R⁸ comprise n-propoxypropyl groups.

99. The tissue defect dressing of claim 71 wherein the heterogeneous crosslinking agent produces crosslinks comprising the following structure: 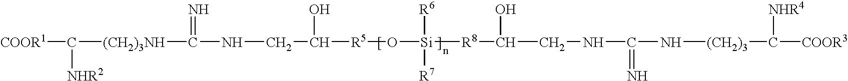
wherein
n is from about 1 to about 50;
R¹ and R² are a remainder of a first keratin molecule;
R³ and R⁴ is a remainder of a second keratin molecule; and,
R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

100. The tissue defect dressing of claim 99 wherein at least one of R⁶ and R⁷ is a methyl group.

101. The tissue defect dressing of claim 99 wherein R⁵ and R⁸ are selected from the group consisting of alkyl groups having from about 1 to about 6 carbon atoms and dimethylsiloxy groups.

102. The tissue defect dressing of claim 100 wherein R⁵ and R⁸ are selected from the group consisting of alkyl groups having from about 1 to about 6 carbon atoms and dimethylsiloxy groups.

103. The tissue defect dressing of any of claims 99 wherein R⁵ and R⁸ comprise n-propoxypropyl groups.

104. The tissue defect dressing of any of claims 100 wherein R⁵ and R⁸ comprise n-propoxypropyl groups.

105. The tissue defect dressing of claim 71 wherein the heterogeneous crosslinking agent produces crosslinks comprising the following structure: 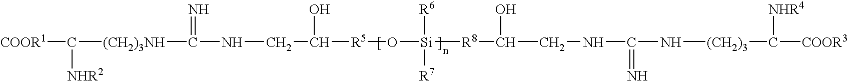
wherein
R¹ and R² are a remainder of a first keratin molecule;
R³ and R⁴ is a remainder of a second keratin molecule; and,
R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

106. The tissue defect dressing of claim 105 wherein at least one of R⁶ and R⁷ is a methyl group.

107. The tissue defect dressing of claim 105 wherein R⁵ and R⁸ are selected from the group consisting of alkyl groups having from about 1 to about 6 carbon atoms and dimethylsiloxy groups.

108. The tissue defect dressing of claim 106 wherein R⁵ and R⁸ are selected from the group consisting of alkyl groups having from about 1 to about 6 carbon atoms and dimethylsiloxy groups.

109. The tissue defect dressing of any of claims 105 wherein R⁵ and R⁸ comprise n-propoxypropyl groups.

110. The tissue defect dressing of any of claims 106 wherein R⁵ and R⁸ comprise n-propoxypropyl groups.

111. The tissue defect dressing of claim 71 wherein the heterogeneous crosslinking agent produces crosslinks comprising the following structure: 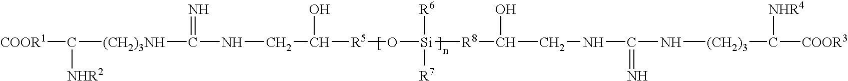
wherein
n is from about 1 to about 50;
R¹ and R² are a remainder of a first keratin molecule;
R³ and R⁴ is a remainder of a second keratin molecule; and,
R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

112. The tissue defect dressing of claim 111 wherein at least one of R⁶ and R⁷ is a methyl group.

113. The tissue defect dressing of claim 111 wherein R⁵ and R⁸ are selected from the group consisting of alkyl groups having from about 1 to about 6 carbon atoms and dimethylsiloxy groups.

114. The tissue defect dressing of claim 112 wherein R⁵ and R⁸ are selected from the group consisting of alkyl groups having from about 1 to about 6 carbon atoms and dimethylsiloxy groups.

115. The tissue defect dressing of claim 111 wherein R⁵ and R⁸ comprise n-propoxypropyl groups.

116. The tissue defect dressing of claim 112 wherein R⁵ and R⁸ comprise n-propoxypropyl groups.

117. The tissue defect dressing of claim 71 wherein the heterogeneous crosslinking agent produces crosslinks comprising the following structure: 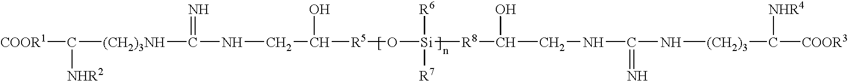
wherein
n is from about 1 to about 50;
R¹ and R² are a remainder of a first keratin molecule;
R³ and R ⁴is a remainder of a second keratin molecule; and,
R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

118. The tissue defect dressing of claim 117 wherein at least one of R⁶ and R⁷ is a methyl group.

119. The tissue defect dressing of claim 117 wherein R⁵ and R⁸ are selected from the group consisting of alkyl groups having from about 1 to about 6 carbon atoms and dimethylsiloxy groups.

120. The tissue defect dressing of claim 118 wherein R⁵ and R⁸ are selected from the group consisting of alkyl groups having from about 1 to about 6 carbon atoms and dimethylsiloxy groups.

121. The tissue defect dressing of claim 117 wherein R⁵ and R⁸ comprise n-propoxypropyl groups.

122. The tissue defect dressing of claim 118 wherein R⁵ and R⁸ comprise n-propoxypropyl groups.

123. The tissue defect dressing of claim 70 wherein the heterogeneous crosslinking agent produces crosslinks comprising the following structure: 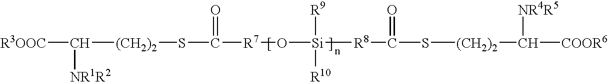
wherein
n is from about 1 to about 50;
R¹, R², and R³ are a remainder of a first protein molecule;
R⁴, R⁵, and R⁶ are a remainder of a second protein molecule; and,
R⁷, R⁸, R⁹ and R¹⁰ are selected from the group consisting of cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality;
carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

124. The tissue defect dressing of claim 123 wherein at least one of R⁹ and R¹⁰ is a methyl group.

125. The tissue defect dressing of claim 123 wherein R⁷ and R⁸ are selected from the group consisting of alkyl groups having from about 1 to about 6 carbon atoms and dimethylsiloxy groups.

126. The tissue defect dressing of claim 124 wherein R⁷ and R⁸ are selected from the group consisting of alkyl groups having from about 1 to about 6 carbon atoms and dimethylsiloxy groups.

127. The tissue defect dressing of claim 123 wherein R⁷ and R⁸ comprise n-propoxypropyl groups.

128. The tissue defect dressing of claim 124 wherein R⁷and R⁸ comprise n-propoxypropyl groups.

129. The tissue defect dressing of claim 71 wherein the heterogeneous crosslinking agent produces crosslinks comprising the following structure: 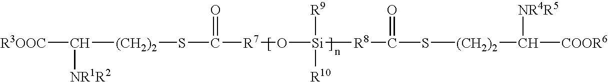
wherein
n is from about 1 to about 50;
R¹, R², and R³ are a remainder of a first protein molecule;
R⁴, R⁵, and R⁶ are a remainder of a second protein molecule; and,
R⁷, R⁸, R⁹ and R¹⁰ are selected from the group consisting of cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

130. The tissue defect dressing of claim 129 wherein at least one of R⁹ and R¹⁰ is a methyl group.

131. The tissue defect dressing of claim 129 wherein R⁷ and R⁸ are selected from the group consisting of alkyl groups having from about 1 to about 6 carbon atoms and dimethylsiloxy groups.

132. The tissue defect dressing of claim 130 wherein R⁷ and R⁸ are selected from the group consisting of alkyl groups having from about 1 to about 6 carbon atoms and dimethylsiloxy groups.

133. The tissue defect dressing of any of claims 129 wherein R⁷ and R⁸ comprise n-propoxypropyl groups.

134. The tissue defect dressing of any of claims 130 wherein R⁷ and R⁸ comprise n-propoxypropyl groups.

135. The tissue defect dressing of claim 71 wherein the heterogeneous crosslinking agent produces crosslinks comprising the following structure: 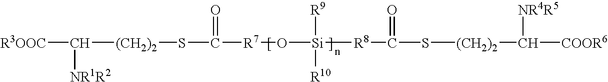
wherein
n is from about 1 to about 50;
R¹, R², and R³ are a remainder of a first protein molecule;
R⁴, R⁵, and R⁶ are a remainder of a second protein molecule; and,
R⁷, R⁸, R⁹ and R¹⁰ are selected from the group consisting of cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

136. The tissue defect dressing of claim 135 wherein at least one of R⁹ and R¹⁰ is a methyl group.

137. The tissue defect dressing of claim 135 wherein R⁷ and R⁸ are selected from the group consisting of alkyl groups having from about 1 to about 6 carbon atoms and dimethylsiloxy groups.

138. The tissue defect dressing of claim 136 wherein R⁷ and R⁸ are selected from the group consisting of alkyl groups having from about 1 to about 6 carbon atoms and dimethylsiloxy groups.

139. The tissue defect dressing of claim 135 wherein R⁷ and R⁸ comprise n-propoxypropyl groups.

140. The tissue defect dressing of claim 136 wherein R⁷ and R⁸ comprise n-propoxypropyl groups.

141. The tissue defect dressing of claim 71 wherein the heterogeneous crosslinking agent produces crosslinks comprising the following structure: 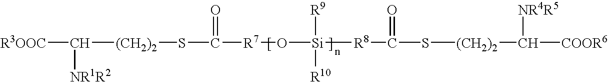
wherein
n is from about 1 to about 50;
R¹, R², and R³ are a remainder of a first protein molecule;
R ⁴, R⁵, and R⁶ are a remainder of a second protein molecule; and,
R⁷, R⁸, R⁹ and R¹⁰ are selected from the group consisting of cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

142. The tissue defect dressing of claim 141 wherein at least one of R⁹ and R¹⁰ is a methyl group.

143. The tissue defect dressing of claim 141 wherein R⁷ and R⁸ are selected from the group consisting of alkyl groups having from about 1 to about 6 carbon atoms and dimethylsiloxy groups.

144. The tissue defect dressing of claim 142 wherein R⁷ and R⁸ are selected from the group consisting of alkyl groups having from about 1 to about 6 carbon atoms and dimethylsiloxy groups.

145. The tissue defect dressing of claim 141 wherein R⁷ and R⁸ comprise n-propoxypropyl groups.

146. The tissue defect dressing of claim 142 wherein R⁷ and R⁸ comprise n-propoxypropyl groups.

147. The tissue defect dressing of claim 71 wherein the heterogeneous crosslinking agent produces crosslinks comprising the following structure: 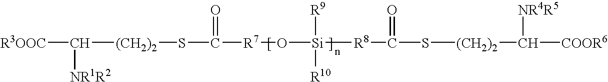
wherein
n is from about 1 to about 50;
R¹, R², and R³ are a remainder of a first protein molecule;
R⁴, R⁵, and R⁶ are a remainder of a second protein molecule; and,
R⁷, R⁸, R⁹ and R¹⁰ are selected from the group consisting of cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

148. The tissue defect dressing of claim 147 wherein at least one of R⁹ and R¹⁰ is a methyl group.

149. The tissue defect dressing of claim 147 wherein R⁷ and R⁸ are selected from the group consisting of alkyl groups having from about 1 to about 6 carbon atoms and dimethylsiloxy groups.

150. The tissue defect dressing of claim 148 wherein R⁷ and R⁸ are selected from the group consisting of alkyl groups having from about 1 to about 6 carbon atoms and dimethylsiloxy groups.

151. The tissue defect dressing of claim 148 wherein R⁷ and R⁸ comprise n-propoxypropyl groups.

152. The tissue defect dressing of claim 149 wherein R⁷ and R⁸ comprise n-propoxypropyl groups.

153. The tissue defect dressing of claim 38 wherein at least one of a moiety selected from the group consisting of the first functional groups, the first reactive pendant groups, they second functional groups, and the second reactive pendant groups is comprises a reactive vinyl group.

154. The tissue defect dressing of claim 41 wherein at least one of a moiety selected from the group consisting of the first functional groups, the first reactive pendant groups, they second functional groups, and the second reactive pendant groups is comprises a reactive vinyl group.

155. The tissue defect dressing of claim 42 wherein at least one of a moiety selected from the group consisting of the first functional groups, the first reactive pendant groups, they second functional groups, and the second reactive pendant groups is comprises a reactive vinyl group.

156. The tissue defect dressing of claim 43 wherein at least one of a moiety selected from the group consisting of the first functional groups, the first reactive pendant groups, they second functional groups, and the second reactive pendant groups is comprises a reactive vinyl group.

157. The tissue defect dressing of claim 44 wherein at least one of a moiety selected from the group consisting of the first functional groups, the first reactive pendant groups, they second functional groups, and the second reactive pendant groups is comprises a reactive vinyl group.

158. The tissue defect dressing of claim 51 wherein at least one of a moiety selected from the group consisting of the first functional groups, the first reactive pendant groups, they second functional groups, and the second reactive pendant groups is comprises a reactive vinyl group.

159 The tissue defect dressing of claim 52 wherein at least one of a moiety selected from the group consisting of the first functional groups, the first reactive pendant groups, they second functional groups, and the second reactive pendant groups is comprises a reactive vinyl group.

160. The tissue defect dressing of claim 38 wherein said first reactive functionality and said second reactive functionality comprise the same reactive moiety.

161. The tissue defect dressing of claim 41 wherein said first reactive functionality and said second reactive functionality comprise the same reactive moiety.

162. The tissue defect dressing of claim 42 wherein said first reactive functionality and said second reactive functionality comprise the same reactive moiety.

163. The tissue defect dressing of claim 43 wherein said first reactive functionality and said second reactive functionality comprise the same reactive moiety.

164. The tissue defect dressing of claim 44 wherein said first reactive functionality and said second reactive functionality comprise the same reactive moiety.

165. The tissue defect dressing of claims 51 wherein said first reactive functionality and said second reactive functionality comprise the same reactive moiety.

166. The tissue defect dressing of claim 52 wherein said first reactive functionality and said second reactive functionality comprise the same reactive moiety.

167. The tissue defect dressing of claim 83 wherein the heterogeneous crosslinking agent produces crosslinks comprising the following structure: 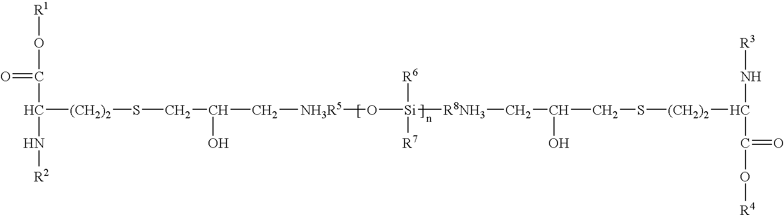
wherein
n is from about 1 to about 50;
R¹ and R² are a remainder of a first keratin molecule;
R³ and R⁴ is a remainder of a second keratin molecule; and,
R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

168. The tissue defect dressing of claim 167 wherein at least one of R⁶ and R⁷ is a methyl group.

169. The tissue defect dressing of claim 167 wherein R⁵ and R⁸ are selected from the group consisting of alkyl groups having from about 1 to about 6 carbon atoms and dimethylsiloxy groups.

170. The tissue defect dressing of claim 168 wherein R⁵ and R⁸ are selected from the group consisting of alkyl groups having from about 1 to about 6 carbon atoms and dimethylsiloxy groups.

171. The tissue defect dressing of claim 167 wherein R⁵ and R⁸ comprise n-propoxypropyl groups.

172. The tissue defect dressing of claim 168 wherein R⁵and R⁸ comprise n-propoxypropyl groups.

173. The tissue defect dressing of claim 83 wherein the heterogeneous crosslinking agent produces crosslinks comprising the following structure: 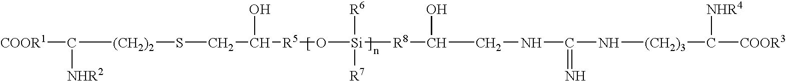
wherein
n is from about 1 to about 50;
R¹ and R² are a remainder of a first keratin molecule;
R³ and R⁴ are a remainder of a second keratin molecule; and,
R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

174. The tissue defect dressing of claim 173 wherein at least one of R⁶ and R⁷ is a methyl group.

175. The tissue defect dressing of claim 173 wherein R⁵ and R⁸ are selected from the group consisting of alkyl groups having from about 1 to about 6 carbon atoms and dimethylsiloxy groups.

176. The tissue defect dressing of claim 174 wherein R⁵ and R⁸ are selected from the group consisting of alkyl groups having from about 1 to about 6 carbon atoms and dimethylsiloxy groups.

177. The tissue defect dressing of claim 173 wherein R⁵ and R⁸ comprise n-propoxypropyl groups.

178. The tissue defect dressing of claims 174 wherein R⁵ and R⁸ comprise n-propoxypropyl groups.

179. The tissue defect dressing of claim 83 wherein the heterogeneous crosslinking agent produces crosslinks comprising the following structure: 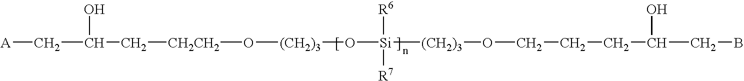
wherein
n is from about 1 to about 50; and
A and B are the remainder of first and second keratin molecules.

180. The tissue defect dressing of claim 179 wherein at least one of R⁶ and R⁷ is a methyl group.

181. The tissue defect dressing of claim 83 wherein the heterogeneous crosslinking agent produces crosslinks comprising the following structure: 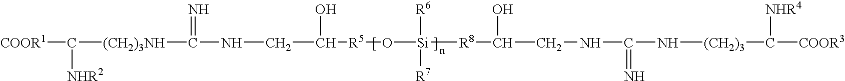
wherein
n is from about 1 to about 50;
R¹ and R² are a remainder of a first keratin molecule;
R³ and R⁴ is a remainder of a second keratin molecule; and,
R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

182. The tissue defect dressing of claim 181 wherein at least one of R⁶ and R⁷ is a methyl group.

183. The tissue defect dressing of claim 181 wherein R⁵ and R⁸ are selected from the group consisting of alkyl groups having from about 1 to about 6 carbon atoms and dimethylsiloxy groups.

184. The tissue defect dressing of claim 182 wherein R⁵ and R⁸ are selected from the group consisting of alkyl groups having from about 1 to about 6 carbon atoms and dimethylsiloxy groups.

185. The tissue defect dressing of claim 181 wherein R⁵ and R⁸ comprise n-propoxypropyl groups.

186. The tissue defect dressing of claim 182 wherein R⁵ and R⁸ comprise n-propoxypropyl groups.

187. The tissue defect dressing of claim 83 wherein the heterogeneous crosslinking agent produces crosslinks comprising the following structure: 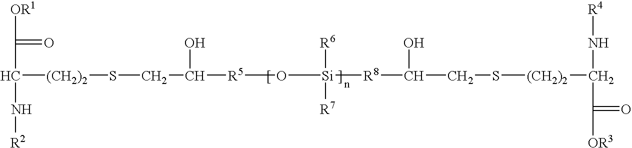
wherein
n is from about 1 to about 50;
R¹ and R² are a remainder of a first keratin molecule;
R³ and R⁴ is a remainder of a second keratin molecule; and,
R⁵, R⁶, R⁷, and R⁸ are reacted groups selected from the group consisting of hydrogen; cyclic, linear, and branched alkyl and heteroalkyl groups having from about 1 to about 6 carbon atoms, said groups comprising both unsubstituted groups and groups substituted with at least one reactive functionality, wherein said heteroalkyl groups comprise one or more heteroatoms selected from the group consisting of nitrogen, oxygen, and sulfur; cyclic, linear, and branched alkenyl and heteroalkenyl groups having from about 2 to about 6 carbon atoms, and mercapto functionalized versions thereof and resonance hybrids thereof, said groups comprising both unsubstituted groups and groups substituted with at least one reactive functionality; carboxyl groups and salts, esters, and amides thereof comprising cyclic, linear, and branched alkyl groups, heteroalkyl groups, alkenyl groups, and heteroalkenyl groups having from about 1 to about 6 carbon atoms wherein said hetero groups comprise one or more heteroatoms selected from the group consisting of nitrogen, oxygen, and sulfur; aromatic groups; alkanols and alkenols having from about 1 to about 6 carbon atoms; alkanolamides and alkenol amides having from about 1 to about 6 carbon atoms; and combinations thereof; alkoxy groups comprising one or more alkyl moieties having a total of from about 1 to about 6 carbon atoms, hydrido groups, and hydroxyl groups.

188. The tissue defect dressing of claim 187 wherein at least one of R⁶ and R⁷ is a methyl group.

189. The tissue defect dressing of claim 187 wherein R⁵ and R⁸ are selected from the group consisting of alkyl groups having from about 1 to about 6 carbon atoms and dimethylsiloxy groups.

190. The tissue defect dressing of claim 188 wherein R⁵ and R⁸ are selected from the group consisting of alkyl groups having from about 1 to about 6 carbon atoms and dimethylsiloxy groups.

191. The tissue defect dressing of claim 187 wherein R⁵and R⁸ comprise n-propoxypropyl groups.

192. The tissue defect dressing of claim 188 wherein R⁵ and R⁸ comprise n-propoxypropyl groups.
